(19) United States
(12) Patent Application Publication
(10) Pub. No.: US 2005/0002981 A1
(43) Pub. Date: Jan. 6, 2005

(54) Medical device

(75) Inventors: Mika Lahtinen, Uppsala (SE); Mikko Laukkanen, Haapalahti (FI); Seppo Yla-Herttuala, Vuorela (FI); Olli-Pekka Leppanen, Uppsala (SE)

Correspondence Address: BURNS DOANE SWECKER & MATHIS L L P, POST OFFICE BOX 1404, ALEXANDRIA, VA, 22313-1404, US

(73) Assignee: FIT Biotech Oy Plc

(21) Appl. No.: 10/693,905

(22) Filed: Oct. 28, 2003

(30) Foreign Application Priority Data
Apr. 30, 2002 (WO) PCT/SE02/00848
Apr. 30, 2001 (FI) 20010898

(51) Int. Cl. A61K 48/00; A61F 2/00

(52) U.S. Cl. 424/423; 514/44

(57) ABSTRACT

The present invention relates to the use of a gene transfer product to reduce hyperplastic connective tissue growth after tissue trauma or implantation of a medical device. The present invention also relates to a medical device with improved biological properties for an at least partial contact with blood, bodily fluids and/or tissues when introduced in a mammalian body, which device comprises a core and a nucleic acid, encoding a product capable of leading to production of extracellular superoxide dismutase present in a biologically compatible medium. Said nucleic acid encodes a translation or transcription product, which is capable of inhibiting hyperplastic connective tissue growth and promoting endothelialisation in vivo at least partially on a synthetic surface of said core. The present invention also relates to a method of producing a medical device according to the invention.

### TECHNICAL FIELD

The invention relates to the use of a gene product for reducing restenosis, increasing endothelialisation and reducing inflammatory reaction. It also relates to the use of said gene product for the reduction of fibrosis and infection rate in a patient. It further relates to a medical device suitable for implantation into a human or animal, such as an implantable prosthetic device, combined with a nucleic acid component, which codes for a gene product that will assist in reducing connective tissue formation. The invention additionally relates to a method of reducing connective tissue and inflammatory reaction formation and to a method for increasing endothelialisation with the purpose of improving a human or animal body's acceptance of a medical device, comprising at least one synthetic surface. Also related to is a method of producing a medical device according to the invention.

### BACKGROUND OF THE INVENTION

Diseased and damaged parts of the body can either be repaired or replaced with several methods. These procedures induce reactive changes in the tissues where the intervention is performed or a device implanted. These reactive changes in tissues are difficult to control and cause complications. Tissue reactive changes occur both in connection to all traumatic handling of tissues, transplantation of biological material or implantation of synthetic material.

To repair tissues either endovascular, endoscopic or surgical methods are performed. All these procedures suffer from reaction to trauma caused by the intervention with following scar tissue formation or fibrotic reaction. In addition to repairing the body part it can also be replaced. Then the donor tissues are generally procured elsewhere: either from the recipient's own body (autograft); from a second donor (allograft); or, in some cases, from a donor of another species (xenograft). Replacement of the body part with native structures is usually preferred method but suffers from tissue reaction in the connection site. Tissue transplantation is costly, and suffers from significant failure rates because of acute inflammatory and long-term fibrotic reactions. Use of artificial or synthetic medical implant devices has been subject of considerable attention, but also this technology suffers from foreign body reaction against the implants with following increase in connective tissue or fibrosis.

Although implant devices can be used in some instances as an alternative to donor-based transplants, they too often produce unsatisfactory results because of the tissue response to trauma, implant's incompatibility with the body, induction of foreign body inflammatory reaction and induction of connective tissue formation with following geometrical changes. Also, lack of cell lining of the cardiovascular device synthetic surface sets up conditions, which increases the risk of thrombosis and other medical/surgical procedural complications. The clinical consequence is either restriction in the flow or occlusion when the device is implanted intravascularly or fibrotic capsule encasing the implant when implanted in the tissues. The final consequence is dysfunction and following other clinical medical complications.

One specific area where the growth factors, genes and implants are used is in the cardiovascular field. Cardiovascular diseases affect a large segment of the human population, and are a cause for significant morbidity, costs and mortality in the society. About 60 million adults in the USA have a cardiovascular disease, which is the major cause of death in the USA. There are one million acute myocardial infarctions or heart attacks per year with 200000 deaths a year. Claudicatio intermittens cause significant morbidity and yearly 150000 lower limb amputations are required for ischemic disease with significant perioperative mortality. Cerebral vascular disease, strokes and bleedings also cause significant morbidity, costs and mortality. There are one million dialysis patients, and yearly 200000 arteriovenous fistula operations are required to surgically create access for dialysis.

Coronary and peripheral vascular diseases are characterised by blockages in the blood vessels providing blood flow and nutrition to the organs. Native blood vessels used as grafts suffer from increased connective tissue formation and accelerated atherosclerosis. This causes subsequently narrowing of the vessel lumen. Other significant disease groups are aneurysms, i.e. local dilatation of the vessels, pseudoaneurysm, and dissection of the vessel wall.

There are pharmacological, surgical and percutaneous strategies to treat these diseases. In pharmacological treatment of ischemic heart disease the goal is to make blood less coagulable, inhibit cholesterol accumulation to the vessel wall and to increase blood flow by vessel dilation or to reduce oxygen consumption.

Alternatively the vessel can be treated with percutaneous transluminal angioplasty (balloon angioplasty), laser angioplasty, atherectomy, roto-ablation, invasive surgery, thrombolysis or a combination of these treatments. The intent of percutaneous methods is to maintain patency after an occluded vessel has been re-opened. Angioplasty suffers from two major problems-abrupt closure and restenosis. Abrupt closure refers to occlusion of a vessel immediately after or within initial hours of dilation procedure. Restenosis refers to re-narrowing of an artery after an initially successful angioplasty. It occurs in 20-40% of patients within the first few months after a successful intervention and is thought to happen because of injury the blood vessels during the balloon inflation. When vessel then heals the smooth muscle cells proliferate faster than endothelial cells narrowing the lumen of the blood vessel (Ip et al. J. Am. College of Cardiol. 1990; 15:1667-1687, Faxonj et al. Am. J. of Cardiology: 1987;60:5B-9B). The percentage of patients that develop early restenosis after balloon angioplasty can be reduced with stent implantation, in which an intraluminal implant such as adjustable stent structural supports, tubular grafts or a combination of them after doing the angioplasty. However, stents actually increase the amount of late luminal narrowing due to intimal hyperplasia, and the overall rate of stent restenosis remains unacceptably high (Kuntz et al. Circ. 2000;101: 2130-2133). These devices suffers from both thrombosis until they are covered with endothelial cells and bleeding complications post-operatively. Because of the risk of thrombosis, anticoagulant therapy is used until the endothelial cell coverage has developed in the stent surface. Endothelial surface does not develop on tubular grafts in humans. Stents and tubular endovascular grafts can also be used to exclude a local vascular dilatation or dissection.

The surgical treatment for cardiovascular disease is to bypass, substitute or reconstruct a diseased vessel with a native or synthetic vascular graft or patch.

All these endovascular and surgical methods are complicated by same problems—trauma to the blood vessel endothelium, formation of excessive connective tissue and inflammatory reaction with following problems with occlusion because of thrombosis or restenosis.

In coronary artery surgery the obstructed vessel is bypassed with an autologous vascular graft. The operation is called CABG, which means coronary artery bypass grafting. In peripheral artery surgery a native or synthetic graft is usually implanted to bypass an obstruction, for example from the groin to the thigh. In some cases arterial segment may alternatively be replaced with a native or synthetic vascular graft. In access surgery for dialysis there is a need for creating an access to clean the blood with the dialysis machine. Usually a connection called fistula is constructed between the upper extremity artery and vein to create a high blood flow required for dialysis. Intracardiac patches are used to repair holes in the cardiac septa or wall. Prosthetic vascular patches are used in vascular surgery in several operations, which requires an incision in the wall of the blood vessel, such as thrombectomies, endarterectomies, aneurysmal repairs and vessel reconstructions. In percutaneous revascularisation catheters with balloons, stents or stent grafts are used to reduce the narrowing or exclude the dilatation or dissection in different anatomical locations such as cerebral, coronary, renal, other peripheral arteries and veins, aorta and in vascular grafts. Balloon dilatations, stents and stent grafts may also be employed in other sites, such as biliary tree, esophagus, bowels, tracheo-bronchial tree and urinary tract.

All endovascular and surgical devices are complicated by same problems—lack of endothelial surfaces on the synthetic surface, formation of excessive connective tissue and inflammatory reaction.

Several strategies have been suggested to improve the patency after vascular interventions and implantation of synthetic vascular implants. About 1 600 000 angioplasties are performed yearly worldwide and stent is inserted vast in majority of these procedures (8th International drug delivery meeting and cardiovascular course on radiation and molecular strategies, Geneva, Switzerland, Feb. 1, 2002). The problem with angioplasty or angioplasty with following stenting is the process of restenosis. Because of the trauma to the vessel an excessive connective tissue formation develops leading to narrowing of the vessel lumen in 20-30 percent of cases after 6 months (Bittl J A: Advances in coronary angioplasty N. Engl. J. Med. 1996; 335:1290-1302, Narins C R, Holmes D R, Topol E J: A call for provisional stenting: the balloon is back! Circulation 1998; 97:1298-1305). The problem with restenosis has been well described in the art and several approaches have been described both scientific literature and patents. Currently there is no strategy in the market to reduce restenosis after simple angioplasty without a device inserted. When using stent devices after angioplasty procedure pharmaceutically coated stents have not been evaluated for long term effects and there is no verified method in humans which would securely reduce long term restenosis rate in stents or stent grafts. The main strategy has been use of various pharmaceutical substances with stents to reduce hyperplasia following the trauma to the tissue such as rapamycin, sirolimus, paclitaxel, tacrolimus, dexamethasone, cytochalasine D and Actinomycin C. One drawback with the current pharmacologically coated devices is the possible disappearance of the effect after the substance has been released from the device surface. Furthermore, the nature of compounds eluting at high local concentration into the vessel wall and downstream vasculature or tissue is an issue of concern. Another drawback is that none of these substances is naturally occurring in the body and thereafter fail to promote natural healing of the foreign body surface. For example are paclitaxel and Actinomycin D cytotoxic to the cells.

In native vessel graft the main problem has been intimal hyperplasia both in the site for connection of the graft and the vessel in the specific body location and the intimal hyperplasia in the graft vessel lumen. The same problem of anastomotic hyperplasia exists when connecting the native vessel with a synthetic vascular graft. More than 350000 synthetic vascular grafts are implanted each year and numerous synthetic biomaterials have been developed as vascular substitutes. As a foreign material, grafts are targets for foreign body reaction and because of thrombogenicity prone to clot in a higher degree than autologous material. To overcome thrombogenicity, most approaches have concentrated on creating a surface that is thromboresistant, with the majority of these efforts being directed toward an improved polymer surface. Studies have demonstrated that selected materials, for example Dacron and ePTFE (expanded polytetrafluorethylene), successfully can be incorporated in both large and small caliber arteries in animal models (Zdrahala, J. Biomater. Appl. 1996; 10:309-29). In humans, Dacron and ePTFE vascular prostheses have met certain clinical success in large and middle-sized arterial reconstructions, but are yet not ideal. However, the success is limited for vessel substitutes smaller than 6 mm in diameter, due to anastomotic hyperplasia i.e., the propensity to develop excessive connective tissue growth in the area where either two native blood vessels or an synthetic artificial blood vessel and an autologous vessel are connected or due to thrombosis (i.e. propensity to develop clots) in the open thrombogenic surface (Nojiri, Artif. Organs 1995 Jan.;19 (1):32-8). The autologous vein grafts suffer from development of stenosis when implanted in arterial position. Gene therapy has been used in prior art to reduce development of restenosis as described in patents and scientific literature. Sleeves impregnated with genes have been described and used as devices around vascular anastomosis to inhibit hyperplasia (WO 98/20027, WO 99/55315). The major drawback in these systems is the cumbersome use of the sleeve and the used substances are growth factors or encoding for a growth factor. Furthermore, studies with thrombogenicity of the implant have been reduced by modifying implant materials or to add chemical compounds to the grafts (e.g. U.S. Pat. No. 5,744,515). The substance mostly used has been heparin, which either is bound to the graft, or is given with a local drug delivery device.

In humans flow surface of foreign implants remains uncovered with endothelial cells except for some case reprots (Wu, J. Vasc. Surg. 1995 May; 21(5):862-7, Guidon, Biomaterials 1993 Jul.; 14(9):678-93). In animals, complete endothelialisation of the vascular graft has been shown to occur in 2-4 weeks depending on species. This period without endothelial surface may result in undesired effects and problems due to e.g. thrombogenicity of the surface. The lack of endothelial surface has led to inferior performance of synthetic grafts compared to autologous grafts (Nojiri, Artif. Organs 1995 Jan;19 (1):32-8). Berger, Ann. of Surg. 1972;175 (1):118-27, Sauvage). Autologous grafts, on the other hand, comprise a step for the harvesting thereof, which leads to longer operation times and also possible complications in the harvesting area. Transposition of omentum with uncompromised vasculature around a porous carotid artery PTFE graft has been demonstrated to increase endothelial cell coverage in the graft lumen in dogs (Hazama, J. of Surg. Res. 1999; 81; 174-180), however, entailing problems with a cumbersome and complex procedure, such as discussed above. Further, grafts have been seeded with endothelial cells, and sodded with endothelial cells or bone marrow (Noishiki, Artif. Organs 1998 Jan; 22(1): 50-62, Williams & Jarrel, Nat. Medicine 1996;2: 32-34). In cell seeding, endothelial cells are mixed with blood or plasma after harvesting and then added to the graft surface during the preclotting period. The endothelial cells used in these methods may be derived from microvascular (fat), macrovascular (for example from harvested veins), or mesothelial sources, whereby the graft later on is implanted. More specifically, these methods comprise several steps, including harvesting of the tissue with endothelial cells, separation of endothelial cells, in some cases a culture of endothelial cells, seeding of endothelial cells on the graft materials and finally implanting the graft. Accordingly, a substantial drawback with these methods is that they are time consuming and cumbersome in practice, and they also require a specific expertise in the area as well as the suitable equipment. Furthermore, such seeded endothelial cells have been genetically engineered, with various results: transduction of the cells with tissue plasminogen activator (tPA) decreases endothelial cell adhesion to the graft surface, and transfection with retrovirus reduces endothelialisation. In order to improve cell seeding, vascular endothelial growth factor (VEGF) transfected endothelial cells or fat cells have been used. In addition to the drawbacks discussed above, this method is even more cumbersome and therefore costly to be useful in practice. A method to transduce endothelial progenitor cells and then re-administer them has been described. However, the problems are still as mentioned above. In order to improve the technology for endothelial cell growth on a surface, ligand treatment of graft surfaces has been suggested. In cell sodding, endothelial cells are administered directly on the polymeric graft surface after harvesting, whereby the graft is implanted, but this technique also includes several steps as mentioned above, which makes it cumbersome as well. Also, tissue engineering, which is also a complex and therefore costly procedure, has been used in order to construct vascular tissues for implantation. Gene technological platform with angiogenetic factors has been suggested to induce endothelial surfaces (PCT/SE 00/02460). Drawback here is that the use of growth factor stimulating effect of growth factors on cells could cause uncontrollably connective tissue growth. Arterial homografts have been described, but they give rise to problems regarding arterial preservation and antigenicity.

Further, worldwide 1 600 000 stenting procedures are performed in the yearly with in average 1,7 stents per patient. Stents, i.e. relatively simple devices of fine network structures, are well known in the art. Stenting for vessel obstruction is usually combined with opening of the artery by dilatation, ablation, atherectomy or laser treatment. These interventions cause trauma and tissue injury to the vessel wall with disruption of the endothelial cell lining. Usually, stents are composed of network of some material, usually stainless steel, which is entered to the diseased area usually percutaneously with a catheter. Stents are of different designs for example, self-deployable/pressure expandable, tubular/conical/bifurcated, permanent/temporary, nondegradable/biodegradable, metal/polymeric material, with or without pharmaceutical compound. They are implanted in a blood vessel in different anatomical locations such as cerebral, coronary, renal, other peripheral arteries and veins, and aorta. Stents may also be used in other locations such as biliary tree, esophagus, bowels, tracheo-bronchial tree and genitourinary tract. Stents may be used for example to treat stenoses, strictures or aneurysms. Stents characteristically have an open mesh construction, or otherwise are formed with multiple openings to facilitate the radial enlargements and reductions and to allow tissue ingrowth of the device structure. After the vessel to dilatation stents have been associated with subacute thrombosis and neointimal thickening leading to obstruction. Before the stent era balloon dilatations alone were used to relieve vessel narrowing. A balloon with hydrogel for delivery of naked DNA coding for VEGF has been described (Riessen, Human Gene Therapy 1993, 4:749-758). U.S. Pat. No. 5,830,879, and van Belle J. Am. Coll. of Cardiol. 1997;29: 1371-9) describes also VEGF plasmid being attached to the balloon with simultaneous deployment of endovascular stent to induce vessel healing and reduce restenosis. Also, a balloon with hydrogel and gene for drug delivery (U.S. Pat. No. 5,674,192, Sahatjian et al.) has been described Catheters have been used to deliver angiogenic peptides, liposomes and viruses with encoding gene to the vascular wall (WO 95125807, U.S. Pat. No. 5,833,651 as above). Catheters have also been used to deliver VEGF protein in order to provide a faster endothelialization of stents (van Belle, Circ. 1997:95 438-448). Further, stent for gene delivery (U.S. Pat. No. 5,843,089, Klugherz B D et al. Nat biotechnology 2000;18: 1181-84) and a stent for viral gene delivery (Rajasubramanian, ASAIO J 1994; 40: M584-89, U.S. Pat. No. 5,833,651) have been described. Endothelial cell seeding on the stent has been used as a method to deliver recombinant protein to the vascular wall, in order to overcome thrombosis, but as mentioned above, this technology is cumbersome and therefore costly.

Stent grafts, also referred to as covered stents, are well known in the art. Such stents are a combination of two parts, namely a stent portion and a graft portion. In a stent graft, a compliant graft is coupled to a radially expandable stent. Stent grafts are considered to be usable, by forming a complete barrier between the stent and the blood flow through the vessel. The graft may serve as a biologically compatible inner covering, by preventing turbulent blood flow over the wire members or other structural materials of which the stent is formed, by preventing thrombotic or immunologic reactions to the metal or to other materials of which the stent is made, and by forming a barrier to separate a diseased or damaged segment of the blood vessel from the blood-flow passing there. In humans, the main problem with stent grafts is the formation of neointimal thickening and lack of complete endotheliahisation leading to occlusion, as discussed above in relation to grafts. Stent grafts may be used in aorta, cerebral, coronary, renal, other peripheral arteries and veins, and aorta Experimental studies have shown that vascular injuries, that arises when the endovascular device is delivered, induces inflammation, local expression and release of mitogens and chemotactic factors, which mediates neointimal lesion formation. Stent grafts may also be used in other locations such as biliary tree, esophagus, bowels, tracheobronchial tree and genitourinary tract.

Thus, at the moment, there is a great need and interest in inhibiting the intimal hyperplasia in the site for the tissue trauma, in the area of device implantation, in the vascular connection site or in a native graft. Also, at the moment, there is a great need and interest to improve the endothelialisation and graft healing in clinical practice. However, hitherto, no such method that works in practice has yet been developed.

Yearly, about 100000 heart valve replacement operations are performed. Heart valve prosthesises are well known in the art. There are of four types of grafts: synthetic grafts, xenografts, allografts and autografts. Xenografts are usually preserved pericardial and porcine valves e.g. Carpentier-Edwards, lonescu-Shiley, Hancock, Pericarbon or stentless valves. Biological degeneration is a major concern in bioprosthetic valves. Degeneration is characterised by disruption of endothelial cell barrier and lack of endothelialisation, increased permeability leading to eased diffusion of circulating host plasma proteins into valve tissue, and increased activity of infiltration processes e.g. calcification and lipid accumulation, and biodegradation of the collagen framework. Also a mild to moderate infiltration of inflammatory cells has been described and studies have shown either no (Isomura J. Cardiovasc. Surg. 1986, 27:307-15) or scarce growth of endothelium on bioprosthetic valve surface (Ishihara, Am. J. Card 1981:48, 443-454) after one year. Changing the method of preservation, neutralisation of glutaraldehyde preservative and pre-endothelialisation of bioprosthetic valves has been suggested to improve valve performance. Some studies have been made on endothelial seeding in this context, but it is clinically cumbersome due to the many steps required, as described above.

As mentioned above, implantable devices are also used in other fields than the cardiovascular. Various implantable devices have been described, such as for structural support, functional support, drug delivery, gene therapy, and cell encapsulation purposes. A variety of devices, which protect tissues or cells producing a selected product from the immune system have been explored for implant in a body, such as extravascular diffusion chambers, intravascular diffusion chambers, intravascular ultrafiltration chambers, and microencapsulated cells. However, when foreign biomaterials are implanted, an inflammatory foreign body reaction starts, which in the end encapsulates the device, and inhibits diffusion of nutritive substances to the cells inside the semipermeable membrane. The zone is nonvascular. The lack of vascularity is an obstacle for diffusion of substances. It decreases long-term viability of the encapsulated endocrine tissue, and it also makes vascular implants more susceptible to infections. The fibrotic capsule without vascularity can also limit the device performance. In U.S. Pat. No. 5,882,354, a chamber holding living cells comprises two zones that by an unknown mechanism prevents the invasion of connective tissue and increases the close vascularisation of the implant.

Some other materials used in the procedure of implanted devices also encounter similar problems as the ones discussed above. For example, suture materials can be mentioned, which materials are used for repair, fixation and/or approximation of body tissues during surgical procedures. Strict requirements exist for sutures for attachment of prosthetic devices or implants regarding strength, biocompatibility and biodegradability.

To summarise, the major drawbacks in this field is the development of excessive connective tissue after endovascular and surgical procedures. For example, after balloon dilatation procedure the result is excessive connective tissue growth with following complications. Also, in the autologous vascular grafts excessive connective growth causes narrowing. In vascular surgical procedures with or without implanted devices the excessive connective tissue formation in the anastomotic areas leads to narrowing of the vessel connection with limitation to the flow and following dysfunction in the organ supplied by that vessel. In vascular implants, when synthetic materials are used, problems also arise due to open thrombotic surfaces where the implant is performed, which in turn generate blood clotting and inferior performance. In synthetic tissue implants, the consequence is a nonvascularised fibrotic non-nutritive zone, which leads to dysfunction of the implant. This together with the inflammatory reaction causes that biocompatibility of the mammalian body, especially the human body, with implanted medical devices cannot be achieved in any satisfactory degree using the prior art methods.

Patent EP1016726 describes use of angiogenetic proteins and genes, such as growth factors (e.g. VEGF) or other genes (e.g. NOS) to create endothelial surfaces after vessel damage and when inserting a stent.

Patent EP1153129 describes use of oligosense nucleotides to inhibit restenosis.

Extracellular superoxide dismutase (EC-SOD) is a secreted antioxidative enzyme, which is widely expressed throughout the body and is the major SOD isoenzyme in plasma. Vessel walls, lung, kidney, thyroid gland and epidymis are shown to be the primary expression sites for EC-SOD.

Patent ES2004687 describes the sequence of EC-SOD. Articles by Li et al. describe use of EC-SOD in myocardial protection (Gene therapy with extracellular superoxide dismutase attenuates myocardial stunning in conscious rabbits. Circulation 1998;98:1438-1448, and Gene therapy with extracellular superoxide dismutase protects conscious rabbits against myocardial infarction. Circulation. 2001;103: 1893-1898).

### SUMMARY OF THE INVENTION

The object of the present invention is to provide a solution to the aforementioned problems. More specifically, one object of the invention is to provide use for a polypeptide or gene transfer product leading to production of extracellular superoxide dismutase (EC-SOD) protein to reduce connective tissue hyperplasia and restenosis after intervention such as angioplasty. Another object of the invention is to provide use for a polypeptide or gene transfer product leading to production of extracellular superoxide dismutase (EC-SOD) protein to reduce connective tissue hyperplasia in autologous or allogenous grafts. Another object of the invention is to provide use for polypeptide or gene transfer product leading to production of extracellular superoxide dismutase (EC-SOD) protein that reduces connective tissue reaction, restenosis and provides endothelialisation of the implants. Another object of invention is to provide use for a polypeptide or gene transfer product leading to production of extracellular superoxide dismutase (EC-SOD) protein that reduces fibrosis both in biological tissues, biological tissue transplants and tissues surrounding the synthetic implants. Another object of the invention is to provide uses for substances that modulate the expression of a gene and production of the protein in order to reduce restenosis and fibrosis. Another objective is to provide use for said gene transfer product in inflammatory vascular and non-vascular disease. Another object of the invention is to provide a medical device, which solves the problems of traumatised vascular tissue reaction resulting in connective tissue hyperplasia. Another object of the present invention is to provide a medical device, which solves the problem with thrombogenic medical implant surfaces resulting in occlusion and other problems. Another object of the present invention is to provide a medical device, which is less cumbersome to use in practice than prior art methods for reducing restenosis or otherwise improving the biocompatibility between foreign materials and the recipient or host thereof. Another object of the invention is to provide a medical device useful in vascular interventions, which entails less risk of being narrowed because of tissue hyperplasia or occluded and reoccluded than hitherto known devices. Yet another object of the invention is to provide a device useful in measurement and control of metabolic functions that is better accepted and maintained in the human or animal body than prior art devices.

Some of the given objects are achieved by administering systemically a protein or gene encoding a translational or transcriptional product being capable of reducing connective tissue formation, inflammatory reaction and promoting endothelialisation. Some of the given objects are achieved by administering locally a protein or gene encoding a translational or transcriptional product being capable of leading to production of extracellular superoxide dismutase (EC-SOD) protein, capable of reducing connective tissue formation, inflammatory reaction and promoting endothelialisation. Some of the above given objects and others are according to the present invention achieved by providing a medical device with improved biological properties for an at least partial contact with blood, bodily fluids and/or tissues when introduced in a mammalian body. Said device comprises a core and a nucleic acid present in a biologically compatible medium and is characterised in that said nucleic acid encodes a translation or transcription product leading to production of extracellular superoxide dismutase (EC-SOD) protein capable of reducing connective tissue formation and promoting endothelialisation in vivo at least partially on a synthetic surface of said core.

In some embodiments, the polypeptide is EC-SOD.

In another embodiments, the nucleic acid encodes EC-SOD protein or polypeptide.

The nucleic acid is present in the biologically compatible medium in naked form, in a viral vector, such as retrovirus, Sendai virus, lenti virus, adeno associated virus, and adenovirus, or in a liposome or is an artificial chromosome.

In another embodiment the nucleic acid is administered systemically.

In another embodiment the nucleic acid is administered locally in the vessel wall.

In another embodiment the nucleic acid is administered locally in the tissue surrounding a device.

In another embodiment, the biologically compatible medium is a biostable polymer, a bioabsorbable polymer, a biomolecule, a hydrogel polymer or fibrin.

In one advantageous embodiment, the nucleic acid is present in a reservoir separate from said core enabling a successive delivery thereof to a mammalian body.

In an alternative embodiment, the nucleic acid has been attached to the core by ionic or covalent bonding.

The synthetic surface is either non-porous or porous, in which case it allows capillary and endothelial cell growth through pores. Preferably, the porosity is from about 0 μm to about 2000 μm.

The present gene transfer product can be used in several different contexts to reduce connective tissue formation e.g. in connection to interventional procedures, implanting a native graft or implanting a medical implant.

The present device is useful in a wide variety of contexts, and may e.g. be a cardiovascular implant, such as an artificial part of a blood vessel, or an endovascular implant In general terms, the present device may be used as an implant used for replacement of part of a mammalian body, where said implant is adapted for an at least partial contact with blood, bodily fluids and/or tissues. Further, the present device is useful as a tissue implant or a biosensor. Preferably, the device is selected from the group consisting of vascular grafts, stents, covered stents, graft connectors and biosensors.

The present invention also relates to a method of producing a medical device according to the invention.

Further, the invention relates to methods of reducing connective tissue hyperplasia in mammalian body after an intervention or trauma which method comprises introducing the nucleic acid systemically to the mammalian body. The invention also relates to the method of introducing nucleic acid to autologous or allogenic graft in a biologically compatible medium, said administration of nucleic acid being performed before, simultaneously as or after the introduction of the device in the body. The invention also relates to the method of introducing a device comprising a an autologous, allogenic and xenogenic synthetic surface in the body with an at least partial contact with blood, bodily fluids and/or tissues and administering a nucleic acid present in a biologically compatible medium to the surroundings thereof. The method is characterised in that the nucleic acid encodes or increases expression of a translation or transcription product of EC-SOD capable of reducing connective tissue growth and inflammatory reaction and promoting endothelialisation and biocompatibility in vivo at least partially on said synthetic surface, said administration of nucleic acid being performed before, simultaneously as or after the introduction of the device in the body.

According to a further aspect of the invention, an use of EC-SOD gene/cDNA or EC-SOD protein is provided for the manufacture of a medicament for the treating of conditions caused by damages due to vascular manipulations, such as restenosis or blood vessel thickening.

Further details regarding the method of treatment are disclosed below and in the appended claims. The method may include administering of the nucleic acid at least once, depending on the case in question.

### BRIEF DESCRIPTION OF THE FIGURE

FIG. 1 shows the histological analysis of serial sections from abdominal aorta two weeks after gene/DNA transfer.

### DEFINITIONS

Below, explanations are provided as to the meaning of some of the terms used in the present specification. Terms that are not specifically defined herein are to be interpreted by the general understanding thereof within the relevant technical field.

It must be noted that as used in this specification and the appended claims, the singular forms “a”, “an”, and “the” include plural referents unless the context clearly dictates otherwise A “restenosis” is here referred to as growth of connective tissue after performing a dilating procedure with or without an implant leading to connective tissue growth in the tubular structure with following narrowing of the tubular structure. The connective tissue growth may occur at any site in the body leading to narrowing of the tubular structure. The connective tissue growth comprises of either increase in some cell type in the area or increase in the volume or the constitutients of extcacellular matrix.

A “fibrosis” is here referred to as growth of connective tissue and formation of an acellular or avascular layer either in an allogenic, autologous or xenogenic biological implant or around a synthetic implant.

“Hyperplastic connective tissue reaction” here defines the reaction leading to an increase of number of connective tissue cells and/or an increase in the volume of extracellular matrix in the tissue, excluding tumour formation, whereby the bulk of the connective tissue may be increased.

“Restenosis” and “fibrosis” may be used interchangeably if not specified in another way.

A “medical implant” is here referred to as an implant, a device, scaffold or prosthesis, and is understood as an object that is fabricated for being implanted at least partly in a mammalian. It is intended to be in contact with bodily tissues and fluids providing at least one contacting surface towards the bodily tissues or fluids. A cardiovascular implant is here referred to an implant in a circulatory system, or an implant being connected with the bloodflow, if not specified in any other way. A tissue implant is here referred to as an implant implanted in other bodily tissues or fluids, if not specified in any other way. For example, a medical implant may be an implantable prosthetic device, and more particularly a cardiovascular implant or a tissue implant, as well as a blood-contacting medical implant, a tissue-contacting medical implant, a bodily fluid-contacting medical implant, an implantable medical device, an extracorporeal medical device, an artificial heart, a cardiac assist device, an endoprosthesis medical device, a vascular graft, a stent graft, a heart valve, a cardiovascular patch, a temporary intravascular implant, an annuloplasty ring, a catheter, a pacemaker lead, a biosensor, a chamber for holding living cells, an organ implant, or a bioartificial organ.

An “attached transferable nucleic acid segment” referred to here, represent the wide variety of genetic material, which can be transferred to the tissues surrounding the medical implant. For example, a nucleic acid segment may be a double or single stranded DNA, or it may also be RNA, such as mRNA, tRNA or rRNA, encoding a protein or polypeptide. Optionally the nucleic acid may be in the form of anti-sense. Suitable nucleic acid segments may be in any form, such as naked DNA or RNA, including linear nucleic acid molecules and plasmids, or as a functional insert within the genomes of various recombinant viruses, such as DNA viruses or retroviruses. The nucleic acid segment may also be incorporated in other carriers, such as salts, polymers, liposomes or other viral structures. The attached transferable nucleic acid segment is attached to the medical implant in such a way, that it can be delivered to and taken up by the surrounding tissues.

The term “attached” refers to adsorption, such as physisorption, chemisorption, ligand/receptor interaction, covalent bonding, hydrogen bonding, or ionic bonding of the chemical substance or biomolecule, such as a polymeric substance, fibrin or nucleic acid to the implant.

A “surrounding tissue” here refers to any or all cells, which have the capacity to form or contribute to the formation of hyperplastic connective tissue or fibrotic reaction of the implant surface. Surrounding tissue also refers to any or all cells that have the capacity to form or contribute to the formation of endothelialised surfaces either on biological or synthetic surfaces. This includes various tissues, such as fat, omentum, pleura, pericardium, peritoneum muscle, vessel wall, and fibrous tissue, but the particular type of surrounding tissue is not important as long as the cells are activated in a way that ultimately gives rise to the formation of hyperplastic connective tissue in the of the implant “Surrounding tissue” is also used to refer to those cells that are located within (excluding cells in tissue chambers), are in contact with, or migrate towards the implant. Also, cells that upon stimulation further attract hyperplastic connective tissue cells or endothelial cells are considered to be surrounding tissue, as well as cells or tissues that arrive to the active site of cardiovascular implant connective tissue hyperplasia, tissue implant fibrosis or endothelialisation. “Surrounding tissue” is also used to refer to inflammatory cells that are either present at the implant area or arrive at the perigraft arear after implantation of the implant.

An “endothelium” is a single layer of flattened endothelial cells, which are joined edge-to-edge forming a membrane coveting the inner surface of blood vessels, heart and lymphatics.

“Endothelialisation” is here referred to the growth of endothelial cells on all mammalian tissue or fluid contacting surfaces of a biomaterial, that is used to form a porous or nonporous implant. Endothelialisation of surfaces can occur via longitudinal growth, ingrowth of capillaries and/or capillary endothelial cells through the pores in the implants, or seeding of circulating endothelial cells or endothelial precursor cells. In this disclosure, it will be used interchangeably with the phrase “capillary endothelialisation”, to refer to the growth of endothelial cells on substantially all tissue contacting surfaces of a biomaterial, that is used to form a porous or nonporous implant, unless otherwise specified.

The terms “capillarisation” and “vascularisation” are here understood as the formation of capillaries and microcirculation on the implant surface, and they will be used interchangeably with endothelialisation, unless otherwise specified

“Angiogenesis” and reflections thereof, such as “angiogenic”, are here referred to formation and growth of endothelial cells in the existing mammalian tissue, such as in the surrounding tissue.

A translational or a transcriptional product having “the potential to prevent restenosis and increase endothelialisation” of the medical implant, is here understood as a chemical substance or biomolecule, preferably a hormone, a receptor or a protein, which, as a result of its activity, can reduce formation of excessive connective tissue and induce endothelialisation or capillarisation of the medical implant.

“Porosity” and reflections thereof, such as “pores” and “porous”, are here referred, if not otherwise specified, to a biomaterial having small channels or passages, which start at a first surface and extend substantially through to a second surface of the biomaterial.

“Surface” refers to the interface between the biomaterial and its environment. It is intended to include the use of the word in both its macroscopic sense (e.g. two major faces of a sheet of biomaterial), as well as in its microscopic sense (e.g. lining of pores traversing the material).

The term compartment refers to any suitable compartment, such as for example a vial or a package.

### DETAILED DESCRIPTION OF THE INVENTION

In a first aspect, the present invention relates to administration of a protein or nucleic acid in a biologically compatible medium to a mammalian body, characterised in that said nucleic acid encodes a translational or transcriptional product leading to production of extracellular superoxide dismutase (EC-SOD) protein capable of reducing hyperplastic connective tissue growth in vivo.

In another aspect, the present invention relates to administration of nucleic acid in a biologically compatible medium to an autologous or allogenous graft, characterised in that said nucleic acid encodes a translational or transcriptional product leading to production of extracellular superoxide dismutase (EC-SOD) protein capable of reducing hyperplastic connective tissue growth in vivo.

In another aspect, the present invention relates to administration of nucleic acid in a biologically compatible medium and an implant, characterised that said the nucleic acid encodes a translational or transcriptional product leading to production of extracellular superoxide dismutase (EC-SOD) protein capable of reducing hyperplastic connective tissue growth in vivo in the tissue surrounding the implant.

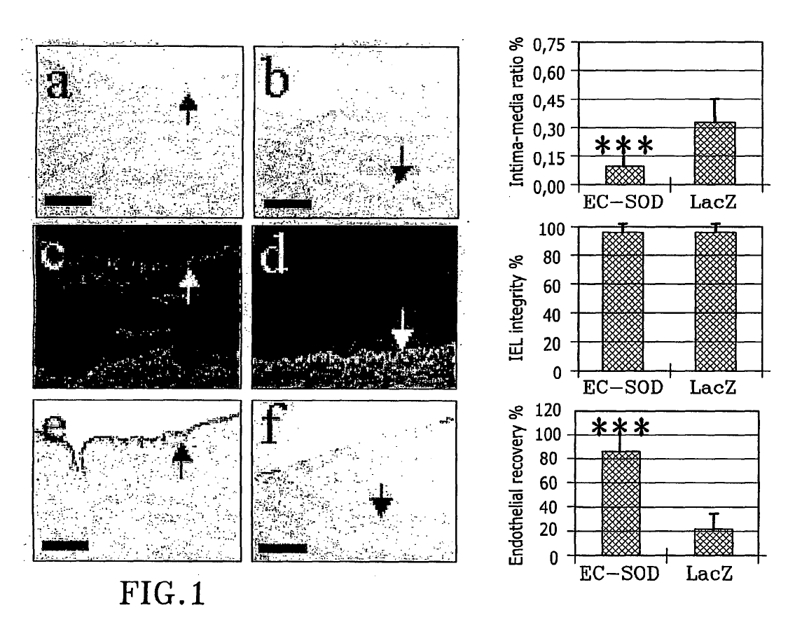

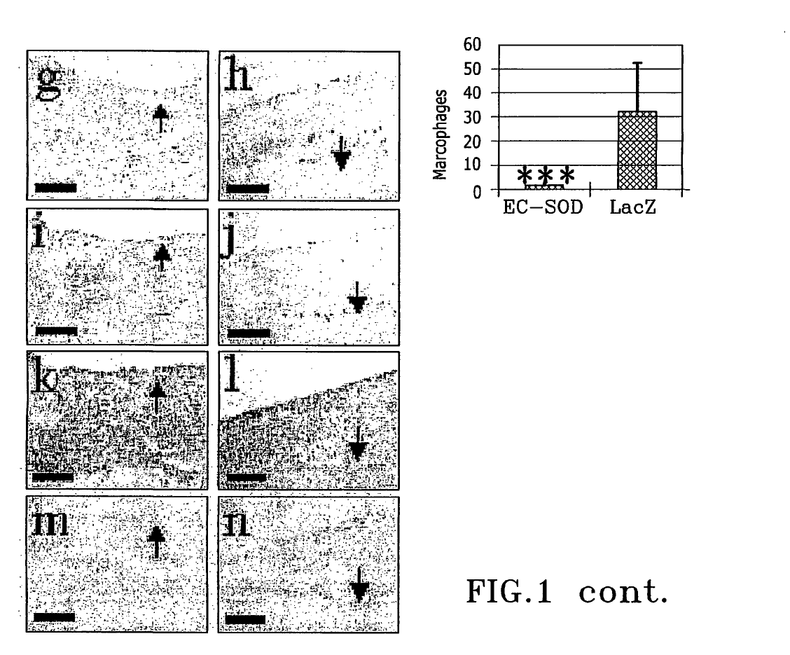

Extracellular superoxide dismutase (EC-SOD) is secreted antioxidative enzyme, which is widely expressed throughout the body and is the major SOD isoenzyme in plasma. Vessel walls, lung, kidney, thyroid gland and epidymis are shown to be the primary expression sites for EC-SOD. About 50% of total SOD amount in human aorta is EC-SOD. In most tissues EC-SOD represents only a minor part of the total SOD activity, which suggests that EC-SOD has a significant physiological role in the redox balance of the vascular wall. Adenovirus mediated EC-SOD gene/cDNA transfer resulted in a significant inhibition of neointima formation in rabbit aortas after balloon denudation (see example and FIG. 1). The therapeutic effect was affecting the whole abdominal aorta, suggesting a systemic effect. EC-SOD appears to be an efficient therapeutic molecule to prevent restenosis.

In another aspect, the present invention relates to a medical device with improved biological properties for an at least partial contact with blood, bodily fluids and/or tissues when introduced in a mammalian body, which device comprises a core and a nucleic acid present in a biologically compatible medium, characterised in that said nucleic acid encodes a translation or transcription product capable of reducing hyperplastic connective tissue reaction and promoting endothelialisation in vivo at least partially on a synthetic surface of said core. The nucleic acid is provided in a way whereby transfer thereof into cells of tissue surrounding the implant is allowed. In the present specification, it is to be understood that the term “introduced in a mammalian body” is used in a broad sense to encompass both devices that are totally included in a body and devices which are only in part introduced, but wherein at least one surface made from a synthetic material is in contact with blood, bodily fluids and/or tissues of said body.

The reduction of hyperplastic connective tissue growth and induction of endothelialisation achieved according to the invention offers many of the advantages of a native structures. Connective tissue hyperplasia comprises of proliferation of cells in the respective tissue and production of extracellular matrix. Endothelium is a single layer of flattened cells, which are joined edge to edge forming a membrane of cells covering the inner surface of blood vessels, heart and lymphatics. In theory, endothelialisation of the graft can occur either via longitudinal growth from the anastomosis area (transanastomotic), ingrowth of capillaries and/or capillary endothelial cells through the synthetic surface, such as a graft wall, and into porosities (transinterstitial), or seeding of circulating endothelial precursor cells. In the transinterstitial migration through the pores, the endothelial cells originate from capillaries through attachment, spreading, inward migration and proliferation.

Thus, even though efforts have been made in the prior art to avoid restenosis and the resulting narrowing of biological tubular structures and connections between polymeric surfaces and bodies own vessels, such efforts have not proved satisfactory with smaller vessels, wherein hyperplasia and thrombosis have caused substantial problems. Also, several efforts have been made in prior art to reduce thrombogenicity without result. Surprisingly, the present invention provides a gene transfer product, which reduces restenosis in biological tissues such as blood vessels after an interventional procedure, and the present invention also provides a novel device, which is protected from restenosis. The present invention provides a versatile technology useful with a large range of implants, and surprisingly also efficient with small size synthetic vessel sections and intravascular implants that have previously been known to develop connective tissue hyperplasia and occlude. The reduction in restenosis achieved according to the invention has not been observed to form in humans in long term studies according to the prior art. The present invention also provides a gene transfer product which increases endothelialisation in traumatatised tissues and on implant surfaces.

In one embodiment the nucleic acid is given systemically and the elevated levels of EC-SOD in blood circulation cause the reduction in restenosis and the increase in endothelialisation.

In one embodiment of the device according to the invention, the nucleic acid is present in the biologically compatible medium associated with adenovirus. EC-SOD has not been described to be used to inhibit restenosis. In an alternative embodiment, the nucleic acid has been introduced in another viral vector selected from the group consisting of retrovirus, lentivirus, Sendai virus and adeno-associated virus. In another embodiment the nucleic acid is present as naked DNA. In yet another embodiment, the nucleic acid is present in a liposome.

The use of gene transfer has been postulated for the treatment or prevention of diseases in several publications. Gene therapy entails the use of genetic material as the pharmacological agent. While originally recognised as a means for treating hereditary diseases, gene therapy is now understood as a powerful tool for delivering therapeutic mRNA or proteins for local and/or systemic use. There are two approaches in gene therapy: ex vivo and in vivo. In the ex vivo approach, cells removed from the host are genetically modified in vitro before they are returned to the host, and in the in vivo approach the genetic information itself is transferred directly to the host without employing any cells as a vehicle for transfer. The gene can be targeted depending on where they are needed, either in stem cells or in situ. The principle for gene therapy is that the cell functions are regulated through the alteration of the transcription of genes and the production of a gene transcription product, such as a polynucleotide or a polypeptide. The polynucleotide or the polypeptide then interacts with other cells to regulate the function of that cell. This transcription change is accomplished with gene transfer. Losordo et al. Circulation 1994, 89:785-792 have shown that gene products that are secreted may have profound biological effects even when the number of transduced cells remains low in contrast to genes that do not encode a secretory signal. For genes expressing an intracellular gene product a much larger cell population might be required for that intracellular gene product to express its biological effects and subsequently more efficient transfection may be required (Isner et al., Circulation, 1995, 91:2687-2692). To illustrate the use of gene therapy this far, genes have e.g. been transferred to adipocytes having a particular utility with respect to diseases or conditions that can be treated directly by in vivo gene transfer to adipocytes. Transfer of nucleic acids into bone tissue has been shown in situ and the use of infected mesothelium either in situ or after isolation as therapeutic resource has also been described.

An extremely wide variety of genetic materials can be transferred to the surrounding tissues using the compositions and methods of invention. For example, the nucleic acid may be DNA (double or single stranded) or RNA (e.g. mRNA, tRNA, rRNA). It may also be a coding nucleic acid, i.e. one that encodes a protein or a polypeptide, or it may be an antisense nucleic acid molecule, such as anti-sense RNA or DNA, that may function to disrupt to gene expression. Alternatively, it may be an artificial chromosome. Thus, the nucleic acids may be genomic sequences, including exons or introns alone, or exons and introns, or coding DNA regions, or any construct that one desires to transfer to the tissue surrounding the prosthesis to reduce restenosis, fibrosis and inflammation or promote endothelialisation. Suitable nucleic acids may also be virtually any form, such as naked DNA or RNA, including linear nucleic acid molecules and plasmids, or a functional insert within the genomes of various recombinant viruses, including viruses with DNA genomes, and retroviruses. The nucleic acid may also be incorporated in other carriers, such as liposomes and other viral structures. The nucleic acid backbone may also be altered or replaced in order to modify the properties such as stability or transfection efficacy.

Chemical, physical, and viral mediated mechanisms are used for gene transfer. Several different vehicles are employed in gene transfer. There are a number of viruses, live or inactive, including recombinant viruses, that can be used to deliver a nucleic acid to the tissues, such as retroviruses, lentivirus, adenoviruses (e.g. U.S. Pat. No. 5,882,887, U.S. Pat. No. 5,880,102) and hemagglutinating viruses of Japan (HVJ or Sendai virus) (U.S. Pat. No. 5,833,651). Retroviruses have several drawbacks in vivo which limit their usefulness. They provide a stable gene transfer, but current retroviruses are unable to transduce nonreplicating cells. The potential hazards of transgene incorporation into the host DNA are not warranted if short-term gene transfer is sufficient. Replication deficit adenoviruses are highly efficient and are used in a wide variety of applications. The adenovirus enters the cell easily through receptor interactions, which has been used as a means for transporting macromolecules into the cell. Non-viral nucleic acids can be packaged within the adenovirus, either as a substitute for, or in addition to normal adenoviral components. Non-viral nucleic acids can also be either linked to the surface of the adenovirus or in a bystander process co-internalised and taken along as a cargo in the receptor-endosome complex. Adenovirus-based gene transfer does not result in integration of the transgene into the host genome, and is therefore not stable. It also transfects nonreplicable cells, which makes adenovirus as an effective vector. Other examples of used viral vectors are adeno-associated viruses (AAV), herpes viruses, vaccinia viruses, lentivirus, poliovirus, other RNA viruses and influenza virus (Mulligan, Science 1993; 260: 926-32; Rowland, Ann Thorac Surgery 1995, 60:721-728). DNA can also be coupled to other types of ligands promoting its uptake and inhibiting its degradation (e.g. U.S. Pat. Nos. 5,972,900, 5,166,320, 5,354,844, 5,844,107, 5,972,707) or directing it to nuclear localisation (Luo & Saltzman, Nat Biotech; 2000, 18:33-37). It can also be coupled to a so-called cre-lox system (Sauer & Henderson, Proc Natl Acad Sci.; 1988, 85:5166). Naked DNA can also be given and the empirical experience is consistent with that double stranded DNA is minimally immunogenic and is unlikely elicit an immunologic reaction.

Plasmid DNA may be administered either in a simple salt solution referred as naked DNA or complexed with a carrier or an adjuvant. In the latter case nucleic acids can be complexed with polycations, proteins or other polymers, dendrimers, incapsulated or associated with liposomes, or coated on colloidal particles. The traditional chemical gene transfer methods are calcium phosphate co-precipitation, carbohydrates (heparansulfate, chitosan), poloxamers, PEI, DEAE-dextran, polymers (U.S. Pat. No. 5,972,707), and liposome-mediated transfer (for example U.S. Pat. No. 5,855,910, U.S. Pat. No. 5,830,430, U.S. Pat. No. 5,770,220), and the traditional physical methods are microinjection, electroporation (U.S. Pat. No. 5,304,120), iontophoresis, a combination of iontophoresis and electroporation (U.S. Pat. No. 5,968,006), ultrasound and pressure (U.S. Pat. No. 5,922,687) (Luo & Saltzman, Nat Biotech; 2000, 18:33-37, Rowland). Transfection efficiency may be improved by any of the known of pharmaceutical measures recognised by skilled in the art

The invention may be employed to promote expression of EC-SOD in tissues surrounding an implant, and to impart a certain phenotype, and thereby promote prosthesis protection from hyperplastic connective tissue growth or fibrosis. This expression could be increased expression of a gene that is normally expressed (i.e. over-expression), or it could be the expression of a gene that is not normally associated with tissues surrounding the prosthesis in their natural environment. Alternatively, the invention may be used to suppress the expression of a gene which normally inhibits a gene expression i.e. gene suppression may be a way of expressing a gene that encodes a protein that exerts a down-regulatory function.

Thus, the nucleic acid used with the device according to the present invention encode transcription or translation products capable of inhibiting connective tissue hyperplasia and tissue fibrosis and promoting or stimulating endothelialisation in vivo, i.e. it is also an antirestenotic or angiogenic factor. Thus, in all embodiments, the nucleic acid encodes an EC-SOD protein or polypeptide.

In another embodiment EC-SOD protein may be used to instead of using EC-SOD encoding genes. It could be given either as a local or systemic therapy.

In another embodiment, the biologically compatible medium is a biostable polymer, a bioabsorbable polymer, a biomolecule, a hydrogel polymer or fibrin. In a specific embodiment, the medium is a mucin composition.

The synthetic surface of the device according to the invention may be either non-porous or porous. Thus, porous, as well as nonporous, implant materials may be used to produce the device, depending on the implant embodiment. For example, graft porosity has been shown to be of importance in vascular graft endothelialisation in animals (Wesolowski, Thorac Cardiovasc Surgeon 1982;30:196-208, Hara, Am. J. Surg.;1967;113:766-69). In the context of sutures, porous sutures have been described to promote tissue ingrowth into the sutures or promote endothelialisation of the sutures (U.S. Pat. No. 4,905,367, U.S. Pat. No. 4,355,426). In porous grafts, such as vascular grafts, capillary and endothelial cell growth is allowed through pores, and the porosity thereof may be from 0 μm to 2000 μm

In one embodiment, the nucleic acid has been attached to the core by ionic or covalent bonding.

In one advantageous embodiment, the nucleic acid is present in a reservoir separate from said core enabling a successive delivery thereof to a mammalian body. The tissue surrounding an implanted device can e.g. be pleura, pericardium, peritoneum, fascia, tendon, fat, omentum, fibrous, muscle, skin, or any other tissue in which inhibition of hyperplastic connective tissue growth, restenosis and fibrosis are required.

Genes expressing anti-restenotic EC-SOD are then attached to the implant or administered in the tissue surrounding the device. The cells in the surrounding tissue become transfected and inhibit restenosis and result in reduction of connective tissue growth in the tissue, a process that results in less hyperplastic or fibrotic tissue reaction with the earlier described advantages of such a tissue.

The surface of the present device may be treated in a variety of ways, in all or parts thereof, e.g. by coating or adding other pharmaceutical substances, as is discussed in more detail below in the experimental section in the general disclosure of materials and methods. The optimal internodal distance for PTFE grafts has been approximately 60 um.

The present device is useful in a wide variety of contexts and depending on the intended use, it may be made from a biomaterial selected from the group of non-soluble synthetic polymers, metals and ceramics with or without modification of the prosthesis surfaces.

Thus, in one embodiment, the device is an implant made of a biocompatible material selected from the group consisting of metal, titanium, titanium alloys, tin-nickel alloys, shape memory alloys, aluminium oxide, platinum, platinum alloys, stainless steel, MP35N, elgiloy, stellite, pyrolytic carbon, silver carbon, glassy carbon, polymer, polyamide, polycarbonate, polyether, polyester, polyolefin, polyethylene, polypropylene, polystyrene, polyurethane, polyvinyl chloride, polyvinylpyrrolidone, silicone elastomer, fluoropolymer, polyacrylate, polyisoprene; polytetrafluoretylene, rubber, ceramic, hydroxyapatite, human protein, human tissue, animal protein, animal tissue, bone, skin, laminin, elastin, fibrin, wood, cellulose, compressed carbon and glass.

Thus, the device may be a medical implant selected from the group consisting of a blood-contacting medical implant, a tissue-contacting medical implant, a bodily fluid-contacting medical implant, an implantable medical device, an extracorporeal medical device, an endoprosthesis medical device, a vascular graft, an endovascular implant, a pacemaker lead, a heart valve, temporary, intravascular implant, a catheter, pacemaker lead, biosensor or artificial organ. In one specific embodiment, the device is a cardiovascular implant, such as an artificial part of a blood vessel, or an endovascular implant. In general terms, the present device may be used as an implant used for replacement of a part of a mammalian body, where said implant is adapted for an at least partial contact with blood, bodily fluids and/or tissues. Further, the present device is useful as a tissue implant or a biosensor. In alternative embodiments, the present device may be any other bioartificial implant that provides a metabolic function to a host, such as a pump for the delivery of insulin or a biosensor to sense the glucose levels etc.

In fact, the present device may be virtually any one of a variety of devices, which protect tissues or cells producing a selected product from the immune system have been explored for implant in a body, such as extravascular diffusion chambers, intravascular diffusion chambers, intravascular ultrafiltration chambers, and microencapsulated cells. Cells can be derived from other species (xenografts), they can be from the same species but different individuals (allografts), sometimes they are previously isolated from the same individual but are modified (autografts) or are of embryonal origin. Bioartificial implants are designed to provide a needed metabolic function to a host, either by delivering biologically active moieties, such as insulin in diabetes mellitus, or removing harmful substances. Membranes can be hydrophobic, such as PTFE and polypropylene, or hydrophilic, such as PAN/PVC and cuprophane.

More specifically, implants encompassed by the invention include, but are not limited to, cardiovascular devices, such as artificial vascular prosthesis, cardiovascular patches, stent grafts, prosthetic valves, artificial hearts, cardiac assist devices, anastomotic devices, graft connectors, annuloplasty ring, indwelling vascular catheters, pacemaker wires, anti-embolism filters, stents and stent grafts for other indications, and tissue implants, such as chambers holding living cells for implantation, biosensors, surgical suture materials, surgical nets, pledgets and patches, tracheal cannulas, bioartificial organs, surgical implants, plastic surgical implants and orthopedic implants. It is anticipated that the herein described procedures may lead to the development of other artificial organs or devices.

In a second aspect, the invention provides a method for producing an implantable medical device. The device can be formed either by the pretreating of a biomaterial with genes, and then fabricating the device from the treated biomaterial, or by first fabricating the device and then treating the exposed surfaces of the device.

In a third aspect, in general terms, the present invention relates to methods for preventing and treating stenosis, restenosis and promoting endothelialisation and vascularisation. One of the methods comprises administering nucleic acid systemically with a composition in a manner effective to transfer said nucleic acid to a tissue in a manner effective to cause a systemic secretion of the protein inhibiting and inhibiting restenosis formation. The other methods of the invention generally comprise to contact the tissue, surrounding the vascular or tissue implant, with a composition comprising a nucleic acid, in a manner effective to transfer said nucleic acid into the tissue, and to inhibit hyperplastic tissue growth and promote endothelialisation of the vascular grafts, cardiovascular patches, stent grafts, heart valves, indwelling vascular catheters, cardiac assist devices and artificial hearts, or to promote vascularisation of tissue implant surfaces. The tissue may be wrapped around the vascular- or tissue implant—nucleic acid composition before implantation to the body. Alternatively, the nucleic acid sequence-prosthesis composition may be implanted in the tissues, or the nucleic acid may be applied to the implantation site before or after the prosthesis implantation, in order to effect, or promote, nuclear acid transfer into the surrounding tissues in vivo. In the transferring of nucleic acids into surrounding tissues, the preferred method involves to first add the genetic material to the tissue compatible medium, to impregnate the prosthesis with the nucleic acid-medium composition, and then to use the impregnated prosthesis to contact an appropriate tissue site. Alternatively, the tissue compatible medium can first be administered on the implant, and then the nucleic acid is added, whereafter the nucleic acid-prosthesis composition is applied to the implantation site. Alternatively nucleic acid is administered to the tissues surrounding the implant, whereafter the implant is implanted, or the implant is first implanted, whereafter the nucleic acid is administered on the implant or to the tissues surrounding the implant. Also, an impregnated implant can be used in combination with administration of nucleic acid in the tissues surrounding the implant before or after implantation. When surrounding tissue is scarce and have a low amount of cells, the impregnated prosthesis can be surgically wrapped in a tissue of higher cell content before implantation. Some of the cardiovascular implants, such as vascular prosthesis, cardiovascular patch and stent grafts, have a porosity that is high enough to allow growth of endothelial cells through the pores, and some other cardiovascular implants, such as heart valves are non-porous.

More specifically, the method according to the invention for inhibiting restenosis of medical implants by transferring a nucleic acid systemically or to the surrounding tissues may be disclosed as a method of improving a mammalian, e.g. a human, body's acceptance of a synthetic surface, which method comprises introducing a device comprising a synthetic surface in the body with an at least partial contact with blood, bodily fluids and/or tissues and administering a nucleic acid present in a biologically compatible medium to the surroundings thereof. The method is characterised in that the nucleic acid encodes a translation or transcription product capable of inhibiting de novo stenosis or restenosis in vivo, said administration of nucleic acid being performed before, simultaneously as or after the introduction of the device in the body. As discussed above in relation to the device according to the invention, the nucleic acid can e.g. be administered in naked form, in a viral vector such as a retrovirus, a Sendai virus, an adeno-associated virus or an adenovirus, or in a liposome.

Depending on the nature of the device, i.e., the condition of the patient who is to receive the implant, the nucleic acid may encode an EC-SOD protein or a polypeptide or a protein inhibiting the downregulation of EC-SOD production. Also, a substance which promotes EC-SOD production can be used. Also, EC-SOD protein may be administered instead of a nucleic acid.

In one embodiment, the nucleic acid or protein is administered systemically or to the surroundings of the device, i.e. the tissue, before introduction thereof in a mammalian body. Alternatively, the nucleic acid or protein is administered systemically or to such surroundings after the introduction thereof. As the skilled in this field will realise, combinations of such administrations are possible, such as a first administration of a certain amount to the surroundings, the introduction of the device, and thereafter one or more additional administration, either according to a predetermined scheme or depending on the body's acceptance thereof and the rate of growth of the new endothelial layer on the synthetic surface.

In another embodiment, the nucleic acid or protein is administered or attached to the device before introduction thereof in a mammalian body. In a specific embodiment, this is achieved by attaching the nucleic acid or protein to the core by ionic or covalent bonding. This embodiment may if appropriate be combined with the last mentioned above, so as to provide a method wherein the device has been pretreated with protein or nucleic acid, while the tissue surrounding the device is later supplemented with further additions of nucleic acid or protein present in a suitable carrier. Also, the treatment with protein and nucleic acid can be combined in different variations. In one embodiment, which is advantageous due to its simplicity, said carrier is sterile water or a sterile aqueous solution. The proteins and nucleic acids of the invention may also be delivered in any suitable pharmaceutical formulations comprising a pharmaceutically acceptable carrier. Examples include aqueous and non-aqueous sterile injection solutions, which may contain antioxidants, buffers, bacteriostats, bactericidal antibiotics; and aqueous and non-aqueos sterile suspensions, which may include suspending agents and thickening agents. The formulation may be presented in unit dose or multi-dose containers, for example sealed ampoules and vials, and may be stored in a frozen or freeze dried (lyophilized) condition requiring only the addition of the sterile liquid carrier, for example water for injection, immediately prior to use.

In another embodiment, the nucleic acid or protein is administered systemically or to the the native vascular graft, i.e. the tissue, before introduction thereof in a mammalian body. Alternatively, the nucleic acid or protein is administered systemically or to surroundings after the introduction thereof. As the skilled in this field will realise, combinations of such administrations are possible, such as a first administration of a certain amount to the native vascular graft, the introduction of the device, and thereafter one or more additional administration, either according to a predetermined scheme or depending on the body's acceptance thereof and the rate inhibition of intimal hyperplasia.

EC-SOD protein or nucleic acid is administered with a view to preventing or treating de novo stenosis or preventing or treating restenosis. It can however also be used to increase endothelialisation.

In alternative embodiments of the present method, the biologically compatible medium is a biostable polymer, a bioabsorbable polymer, a biomolecule, a hydrogel polymer or fibrin.

The present method may be used in the context of any mammalian, such as in the treatment of humans to reduce excessive connective tissue growth and increase the biocompatibility of a foreign, at least partly synthetic, device, such as a medical implant. Further, the present method may be used in monitoring, where a biosensor or other similar equipment is introduced.

Thus, as mentioned above and as further detailed below, the device used in the present method may be an implant used in cardiovascular surgery, a device replacing a part of the body, such as a vessel, a device for introduction into a human body, such as an endovascular implant, a tissue implant, or a biosensor.

In summary, with respect to the transfer and expression of therapeutic proteins or genes according to the present invention, the ordinary skilled artisan is aware that different genetic signals and processing events control levels of nucleic acids and proteins/peptides in a cell, such as transcription, mRNA translation, and post-translational processing. These steps are affected by various other components also present in the cells, such as other proteins, ribonucleotide concentrations and the like.

Accordingly, in general terms, the present invention concerns anti-stenotic, anti-restenotic and anti-fibrotic treatments and devices, which devices may be generally considered as molded or designed vascular implant-gene compositions. The devices of the invention are naturally a tissue-compatible implant in which one or more anti-restenotic or anti-fibrotic EC-SOD gene or EC-SOD proteins are associated with the implant. The combination of EC-SOD gene or protein and implant components is decided by the skilled in this field in order to render the device capable of inhibiting stenosis, restenosis or fibrosis, or stimulating angiogenesis when implanted. Devices according to the invention may be of virtually any size or shape, so that their dimensions are adapted to fit the implantation site in the body.

As indicated above, EC-SOD proteins or nucleic acids may be used for the treatment or prevention of intimal hyperplasia arising from any clinical circumstances. For example it is possible to treat hyperplasia arising after any type of surgical procedure, including angioplasty, for example balloon angioplasty; by-pass surgery, such as coronary artery by-pass surgery in which a vein is anstomosed to an artery; other anstomosis procedures, for example anastomosis in legs or trachea; endarterectomy, for example carotid en rectomy. It is also possible to treat intimal hyperplasia associated with arterial damage or hypertension, for example pulmonary hypertension. The invention provides for treatment in any type of blood vessel, e.g. in an artery or vein.

According to invention it is possible to treat or ameliorate established intimal hyperplasia or to prevent intimal hyperplasia from arising. Similarly, it is possible to diminish the likelihood of intimal hyperplasia from arising, or to diminish the severity of established intimal hyperplasia or hyperplasia that is likely to arise. Treatment according to the invention may take place before, during, or after surgical procedure, for example to reduce the hyperplasia or increase endothelialisation after the procedure.

The following section is provided to illustrate the present invention and should not be interpreted as limiting the invention in any way. References given below and elsewhere in the present application are hereby included by reference.

This section describes alternative materials and methods that may be utilised in this context in order to offer as many possibilities as possible within the scope of the appended claims. Thereafter, under the headline examples, specific disclosures of the experiment carried out to describe the effect of the invention and the advantages thereof will be provided.

1. EC-SOD as Implant Endothelialisation Increasing or Restenosis Reducing Gene

As used herein, the term “restenosis or fibrosis inhibiting gene and endothelialisation promoting gene” is used to refer to a gene or a DNA coding region that encodes a protein, a polypeptide or a peptide, that is capable of promoting, or assisting in promotion of EC-SOD mediated inhibition of restenosis and fibrosis or EC-SOD mediated endothelialisation or vascularisation, or that decreases the rate of EC-SOD mediated inhibition of restenosis or fibrosis, or increases the rate of EC-SOD mediated endothelialisation or vascularisation, or EC-SOD mediated inhibition of macrophage infiltration. The terms inhibiting and reducing or promoting, inducing and stimulating are used interchangeably throughout this text, to refer to direct or indirect processes that ultimately result in less formation connective tissue to the site of tissue trauma or implantation of device or increase the formation of implant endothelium and/or capillaries, or in an increased rate of endothelialisation and/or capillarisation either with or without implantation of a device. Thus, an implant restenosis or fibrosis inhibiting gene or endothelialisation promoting gene is a gene, which, when it is expressed, causes the phenotype of the cell to change, so that the cell either differentiates, stimulates other cells to differentiate, attracts restenosis inhibiting genes or implant endothelialisation promoting cells, or otherwise functions in a manner that ultimately gives rise to new implant endothelium through an increase in EC-SOD either locally or systemically.

In general terms, a restenosis inhibiting gene or vascular implant endothelialisation promoting gene may also be characterised as a gene capable of reducing formation of connective tissue or capable of stimulating the growth of endothelium in the tissues surrounding vascular prosthesis and thereby reducing restenosis and fibrosis or promoting the endothelialisation or the vascularisation of the traumatised tissue or of the implant through increase in EC-SOD. Thus, in certain embodiments the methods and compositions of the invention may be to stimulate growth of endothelium in vascular prosthesis itself and also in tissues surrounding it.

A variety of anti-restenotic factors are now known, of which all are suitable for use in connection with the present invention. Anti-restenotic genes and their encoding proteins, include, for example, hormones, many different growth factors and cytokines, growth factor receptor genes, enzymes and polypeptides. Examples of suitable anti-restenotic factors include those of the VEGF and FGF-family, TGF-β Type II receptor, NOS and HGF.

The preferred anti-restenotic gene product is EC-SOD. There is a considerable variation in the terminology currently employed in the literature referring to genes and polypeptides. It will be understood by those skilled in the art, that all genes that cause increase in an active EC-SOD protein are considered for use in this invention, regardless of the differing terminology that may be employed.

The DNA sequences for several EC-SOD genes have been described both in scientific articles (Genomics 22; 162-171, 1994, Hjalmarsson et al., Proc. Natl. Acad. Sci. USA 84; 6340-6344,1987. Laukkanen et al., Arteriosclerosis, Thrombosis and Vascular Biology 19; 2171-2178, 1999. Laukkanen et al., Gene, 254, 173-179, 2000), U.S. Pat. No. 5,788,961 and in WO 87/01384.

As disclosed in the above patents, and known to those skilled in the art, the original source of a recombination gene or a DNA to be used in a therapeutic regimen need not be of the same species as the animal to be treated. In this regard, it is contemplated that any recombinant anti-restenotic or anti-fibrotic gene may be employed to reduce excessive connective tissue formation or promote vascular prosthesis endothelialisation in a human subject or an animal, such as e.g., horse. Particularly preferred genes are those from human, as such genes are most preferred for use in human treatment regiments. Recombinant proteins and polypeptides encoded by isolated DNA and genes are often referred to with the prefix r for recombinant and rh for recombinant human.

To prepare an anti-restenotic or anti-fibrotic gene, gene segment or cDNA, one may follow the teachings disclosed herein and also teachings of any of the patents or scientific documents referred to in the reference list or in the scientific literature. For example, one may obtain EC-SOD segments by using molecular biological techniques, such as polymerase chain reaction (PCR), or by screening a cDNA or genomic library, using primers or probes with sequences based on the above nucleotide sequence. The practice of such a technique is a routine matter for those skilled in the art, as taught in various scientific articles, such as Sambrook et al., incorporated herein by reference. The anti-restenotic or anti-fibrotic gene and DNA segments that are particularly preferred for use in the present compositions and methods, is EC-SOD or parts of its coding or non-coding sequence. It is also contemplated that one may clone further genes or cDNA that encode a protein or polypeptide that increases EC-SOD expression and protein production or decreases EC-SOD downregulation. The techniques for cloning DNA, i.e. obtaining a specific coding sequence from a DNA library that is distinct from other portions of DNA, are well known in the art. This can be achieved by, for example, screening an appropriate DNA library. The screening procedure may be based on the hybridisation of oligonucleotide probes, designed from a consideration of portions of the amino acid sequence of known DNA sequences encoding related anti-restenotic proteins. The operation of such screening protocols are well known to those skilled in the art and are described in detail in the scientific literature, for example Sambrook et al. (Sambrook et al., Molecular Cloning: a Laboratory Manual, 1989, Cold Spring Lab Press; Inniste et al., PCR strategies, 1995, Academic Press, New York).

EC-SOD genes, with sequences that vary from those described in the literature, are also encompassed by the invention, as long as the altered or modified gene still encodes a protein that functions to stimulate surrounding tissues of cardiovascular or tissue implants, in any direct or indirect manner. These sequences include those caused by point mutations, those due to the degeneracy of the genetic code or naturally occurring allelic variants, and further modifications that have been introduced by genetic engineering such as a hybrid gene, i.e. by the hand of man.

Techniques for introducing changes in nucleotide sequences that are designed to alter the functional properties of the encoded proteins or polypeptides are well known in the art. Such modifications include the deletion, insertion or substitution of bases, and thus, changes in the amino acid sequence. Changes may be made to increase the EC-SOD activity of a protein, to increase its biological stability or half-life, to decrease its degradation, increase its secretion, change its glycosylation pattern, and the like. All such modifications of the nucleotide sequences are encompassed by this invention.

It will also be understood that one, or more than one, anti-restenotic or anti-fibrotic gene may be used in the methods and compositions of the invention. The nucleic acid delivery may thus entail the administration of one, two, three, or more anti-restenotic or anti-fibrotic genes or proteins. The maximum number of genes or proteins that may be applied is limited only by practical considerations, such as the effort involved in simultaneously preparing a large number of gene constructs or even the possibility of eliciting an adverse cytotoxic effect. The particular combination of genes may be two or more anti-restenotic genes, or it may be such that a growth factor inhibiting gene is combined with a hormone gene. A hormone or growth factor gene may even be combined with a gene encoding a cell surface receptor capable of interacting with a polypeptide product of the first gene. Also, an EC-SOD gene can be combined with genes encoding antisense products intracellular aptamer molecules or ribozymes. In using multiple genes, the genes may be combined on a single genetic construct under control of one or more promoters, or they may be prepared as separate constructs of the same or different types. Thus, an almost endless combination of different genes and genetic constructs may be employed. Certain gene combinations may be designed to, or their use may otherwise result in, achieving synergistic effects on reducing excessive connective tissue formation and fibrosis or angiogenesis and endothelialisation. Any of all those combinations are intended to fall within the scope of the present invention. A person skilled in the art readily would be able to identify likely synergistic gene combinations or gene protein combinations. Another gene may encode a protein that inhibits the growth of neointimal cells, for example inducible nitric oxide synthase (iNOS) or endothelial cell nitric oxide synthase (ecNOS). Proteins or products of enzyme proteins that inhibit thrombosis, e.g. prostacyclin, tissue plasminogen activator (tPA), urokinase, and streptokinase, are also of interest for co-transfection. Also EC-SOD may be combined with other genes, which later inhibit the overexpression of EC-SOD or modulate EC-SOD expression at any level such as transcription or translation. Administration may occur before, simultaneously or after administration of the EC-SOD nucleic acid.

It will also be understood that the nucleic acid or gene could, if desired, be administered in combination with further agents, such as, e.g. proteins, polypeptides, aptamer oligonucleotides, ribozymes, transcription factor decoy oligonucleotides or various pharmacologically active agents, growth factors inhibiting restenosis formation, substances such as heparin to inhibit excessive connective tissue growth etc. Also, immunosuppressants, anti-inflammatory and other anti-restenosis substances may be used. As long as genetic material or protein forms part of the composition, there is virtually no limit for including other components, given that the additional agent does not cause a significant adverse effect upon contact with the target cells or tissues. The nucleic acids or protein may thus be delivered along with various other agents. Also, nucleic acid or protein may be delivered along with an implant giving radiation, ultrasound, and electric current or light energy to the surrounding tissue to excert a specific effect along with anti-fibrosis.

It will also be understood that the nucleic acid or gene can be administered in combination with a simultaneous cell seeding or sodding procedure, or simultaneous administration of stem cells or stimulation of stem cell population at the site of implant. It can also be combined with simultaneous seeding or sodding with genetically modified cells.

Gene Constructs and Nucleic Acid:

As used herein, the terms gene and nucleic acid are both used to refer to a DNA molecule that has been isolated, and are free of total genomic DNA of a particular species. Therefore, a gene or a DNA encoding EC-SOD refers to a DNA that contains sequences encoding an EC-SOD protein, but it is in isolated from, or purified free from, total genomic DNA of the species from which the DNA is obtained. Included within the term DNA are DNA segments and smaller fragments of such segments aptamers, and also recombinant vectors, including for example plasmids, cosmids, artificial chromosomes, phages, lentivirus, retroviruses, adenoviruses, and the like.

The term gene is used for simplicity to refer to a functional protein- or peptide-encoding unit As will be understood by those skilled in the art, this functional term includes both genomic sequences and cDNA sequences. Of course, this refers to the DNA segment as originally isolated, and does not exclude genes or coding regions, such as sequences encoding leader peptides or targeting sequences, later added to the segment by man.

This invention provides novel ways to utilise various EC-SOD protein and known EC-SOD DNA segments and recombinant vectors. Many such vectors are readily available. However, there is no requirement for a highly purified vector to be used, as long as the coding segment employed encodes an EC-SOD protein, and does not include any coding or regulatory sequences that would have an adverse effect on the tissues. Therefore, it will also be understood that useful nucleic acid sequences may include additional- residues, such as additional non-coding sequences flanking either of the 5′ or 3′ portions of the coding region or may include various internal sequences, i.e. introns, which are known to occur within genes.

After the identification of an appropriate EC-SOD encoding gene or DNA molecule, it may be inserted into any one of the many vectors currently known in the art. In that way it will direct the expression and production of the EC-SOD when incorporated into a tissue surrounding the implant. In a recombinant expression vector, the coding portion of the DNA segment is positioned under the control of a promoter. The promoter may be in a form that is naturally associated with an EC-SOD gene. Coding DNA segments can also be positioned under the control of a recombinant, or heterologous, promoter. As used herein, a recombinant or heterologous promoter is intended to refer to a promoter that is not normally associated with an EC-SOD gene in its natural environment. Such promoters may include those normally associated with other anti-restenotic genes, and/or promoters isolated from any other bacterial, viral, eukaryotic, or mammalian cell. Naturally, it will be important to employ a promoter that effectively directs the expression of the DNA segment in tissues. The use of recombinant promoters to achieve protein expression is generally known to those skilled in the art of molecular biology (Sambrook et al.). The promoters used may be constitutive, or inducible, and can be used under the appropriate conditions to direct high level or regulated expression of the introduced DNA segment. The currently preferred constitutive promoters are for example CMV, RSV LTR, immunoglobulin promoter, SV40 promoter alone, and the SV40 promoter in combination with the SV40 enhancer, and regulatable promoters such as the tetracyclin-regulated promoter system, or the metalothionine promoter. The promoters may or may not be associated with enhancers, where the enhancers may be naturally associated with the particular promoter or associated with a different promoter. A termination region is provided 3′ to the EC-SOD coding region, where the termination region may be naturally associated with the cytoplasmic domain or may be derived from a different source. A wide variety of termination regions may be employed without adversely affecting expression. After various manipulations, the resulting construct may be cloned, the vector isolated, and the gene screened or sequenced to ensure the correctness of the construct. Screening can be done with restriction analysis, sequencing or alike.

EC-SOD gene and DNA segments may also be in the form of a DNA insert, which is located within the genome of a recombinant virus, such as, for example, recombinant adenovirus, adenoassociated virus (AAV) or retrovirus. To place the gene in contact with a tissue surrounding an implant, one would, in such embodiments, prepare the recombinant viral particles, the genome that includes the EC-SOD gene insert, and simply contact the tissues surrounding the implant with the virus, whereby the virus infects the cells and transfers the genetic material. In some embodiments of the invention, one would attach virus in a composition to an implant, such as a vascular prosthesis, stent, stent graft or graft connector, and then contact the tissue surrounding the implant with the implant in site. The virus is released from the composition, whereby cells grow into the implant, thereby contacting the virus and allowing viral infection, which results in that the cells take up the desired gene or cDNA and express the encoded protein, which in turn results in inhibition of connective tissue formation.

In a preferred embodiment, the methods of the invention involve to prepare a composition in which the EC-SOD gene is attached to or are impregnated on a vascular prosthesis, stent, a stent graft, a heart valve, a graft connector, or a tissue implant to form a vascular prosthesis-, a stent-, an endovascular graft-, a graft connector-, a heart valve- or a tissue implantgene composition and then the vascular prosthesis-, stent-, stent graft-, graft connector-, heart valve-, tissue implant-gene composition is placed in contact with tissue surrounding the said cardiovascular or tissue implant. Vascular prosthesis-, cardiovascular patch-, stent graft-, heart valve-, graft connector-, tissue implant-gene compositions are all those in which a gene is adsorbed, absorbed, or otherwise maintained in contact with the said implant.

2. Nucleic Acid Transfer Into Cells of Tissue Surrounding an Implanted Device

Once a suitable vascular implant-gene composition has been prepared or obtained, all that is required for delivering the EC-SOD protein or EC-SOD gene to the surrounding tissue, is to place the cardiovascular implant-gene or tissue implant-gene composition surgically, or with the help of a catheter, in contact with the desired site in the body, with or without first wrapping it with the surrounding tissue. The methods are well known to a person skilled in the art. The EC-SOD gene or protein can also be administered systemically into the circulation or to the tissue before, during or after implanting the cardiovascular or tissue implant to the site. This could be an arteriovenous fistula, arterial bypass graft or interposition graft, a venous graft, cardiovascular patch, artificial heart, stent graft, stent, heart valve, cardiac asssist device, anastomotic device, annuloplasty ring, vascular catheter, pacemaker wire, tracheal cannula, biomedical sensor, chamber for living cells, artificial organ, organ implant, orthopedic implant, suture material, surgical patch, clip or pledget, or any medical device, all of which comprise at least one synthetic surface.

In the present invention, one or more vectors are transferred to any surrounding tissue, which preferably is a mammalian tissue. Several publications have postulated the use of gene transfer for the treatment or prevention of diseases (Levine and Friedman, Curr. Opin. in Biotech. 1991; 2: 840-44, Mulligan, Science 1993; 260: 926-32, Crystal, Science 1995; 270:404-410, Rowland, Ann. Thorac Surgery 1995; 60:721-728; Nabel et al., Science 1990; 249: 1285-88). The eukaryotic host cell is optimally present in vivo. According to the present invention, the contacting of cells with the vectors of the present invention can be by any means by which the vectors will be introduced into the cell. Such introduction can be by any suitable method. Preferably, the vectors will be introduced by means of transfection, i.e. using the natural capability of the naked DNA to enter cells (e.g., the capability of the vector to undergo receptor-mediated endocytosis). However, the vectors can also be introduced by any other suitable means, e.g. by transduction, calcium phosphate-mediated transformation, microinjection, electroporation, osmotic shock, and the like.

The method can be employed with respect to various cells, differing both in number of vector receptors as well as in the affinity of the cell surface receptors for the vector. According to the invention, the types of cells to which gene delivery is contemplated in vivo include all mammalian cells, more preferably human cells. The vectors can be made into the compositions appropriate for contacting cells with appropriate (e.g. pharmaceutically acceptable) excipients, such as carriers, adjuvants, vehicles, or diluents. The means of making such a composition, and means of administration, have been described in the art. Where appropriate, the vectors can be formulated into preparations in solid, semisolid, liquid, or aerosol forms, such as aerosol, spray, paste, ointment, gel, glue, powders, granules, solutions, injections, creme and drops, in the usual ways for their respective route of administration without excluding any other method. A pharmaceutically acceptable form, that does not ineffectuate the compositions of the present invention should be employed. In pharmaceutical dosage forms, the compositions can be used alone or in an appropriate association, as well as in combination with other pharmaceutically active compounds. For example, nucleic acids encoding for EC-SOD can be administered together with nucleic acids encoding for inhibiting platelet deposition or smooth muscle cell proliferation. Accordingly, the pharmaceutical composition of the present invention can be delivered via various ways and to various sites in a mammalian to achieve a particular effect. A person skilled in the art will recognise that although more than one way can be used for administration, a particular way can provide a more immediate and more effective reaction than the other way. Systemic delivery can be accomplished for example by administration intravenously, intra-arterially, subcutaneously or intramuscularly. It can also be achieved through mucosal membranes such as nasal mucosa. Local delivery can be accomplished by administration comprising topical application or instillation of the formulation on the implant, or administration of the formulation directly, to the tissues surrounding the implant in vivo, or any other topical application method. Administration of the drug this way, enables the drug to be site-specific, in a way that release of high concentrations and/or highly potent drugs may be limited to direct application to the targeted tissue. When delivering the nucleic acids either systemically or locally they can be delivered in solution in naked form in any biocompatible salt solutions or complexed with any biocompatible substances. Examples of the ways to complex nucleic acids is to use polycations (eg oligodendromer), proteins (eg transferrin) or other polymers (eg DEAE-Dextran, polylysine). Also derivatives and salts of the examples are included. Other example is to encapsulate the nucleic acid or associate with liposomes or coated on colloidal particles. Preferred methods is to deliver nucleic acids in an aqueous solution incorporated in fibrin, hydrogel, glycosaminoglycans, glycopolysaccharides, or any other biocompatible polymeric carrier matrix, such as alginate, collagen, mucin, hyaluronic acid, polyurethane, cellulose, polylactic acid, poloxamer which covers at least a portion of the implant (U.S. Pat. No. 5,833,651). Nucleic acids can be added to the polymer-coated implant, either at the time of implant manufacture or by the physician prior to, during or after implantation. The polymer may also be either a biostable or a bioabsorbable polymer, depending on the desired rate of release or the desired degree of polymer stability. It may be naturally occuring or synthetic compound, also derivatives and salts of the compounds are included. A bioabsorbable polymer is more desirable, as it is supposed to cause no chronic local response. Bioabsorbable polymers that may be used include, but are not limited to, poly(L-actic acid), polycaprolactone, poly(actide-coglycolide), poly(hydroxybutyrate), poly(hydroxybuturate-co-valerate), polydioxanone, polyorthoester, polyanhydride, poly(glycolic acid), poly(D,L-lactic acid), polylactic-polyglycolic acid, polyglactin, polydioxone, polygluconate, poly(glycolic acid-cotrimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, and biomolecules, such as fibrin, fibrinogen, cellulose, starch, collagen, mucin, fibronectin, and hyaluronic acid. Also, biostable polymers with a relatively low chronic tissue response, such as polyurethanes, silicones, and polyesters could be used if they can be dissolved or polymerised on the implant, such as polyelolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidine halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, ethylene-vinyl acetate copolymers: polyamides, such as Nylon 66 and polycaprolactam; alkyd resins; polycarbonates; polyoxymethylenes; polyimides; polyethers; epoxy resins; polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethylcellulose (U.S. Pat. No. 5,776,184). Also fibrin together with other biocompatible polymers, either natural or synthetic and their derivatives and salts, may be used Of the polymers glycopolysaccharides may be advantageous. In one aspect there is a solid/solid solution of polymer and drug. This means that the drug and the polymer both are soluble in the same solvent and have been intimately admixed in the presence of that solvent. The drug and polymer can be applied in various ways, such as by simply immersing the implant into the solution or by spraying the solution onto the implant (U.S. Pat. No. 5,776,184). Various hydrogel polymers can be used, such as those selected from the group consisting of polycarboxylic acids, cellulosic polymers, gelatin, alginate, poly 2-hydroxyethylmethylacrylate (HEMA) polyvinylpyrrolidine, maleic anhydride polymers, polyamids, polyvinyl alcohols, polyethylene oxides, polyethylene glycol, polyacrylamide, polyacids, e.g. polyacrylic acids, polysaccharide, e.g. a mucopolysaccharide such as hyaluronic acid (U.S. Pat. No. 5,674,192 and U.S. Pat. No. 5,843,089). The polymer can be porous or nonporous on the implant. Several layers of polymers can be utilised and several different polymers can be combined on the same implant. Different layers and different polymers can carry different pharmacological substances (U.S. Pat. No. 5,833,651). Also, one or more surfaces of the implant can be coated with one or more additional coats of polymer that is the same or different from the second polymer. The adhesion of the coating and the rate at which the therapeutic compound is delivered can be controlled by selection of an appropriate bioabsorbable or biostable polymer, and by the ratio of therapeutic compound to polymer in the solution (U.S. Pat. No. 5,776,184). The dosage applied to the tissue may also be controlled by regulating the time of presoaking therapeutic compound into the hydrogel coating to determine the amount of absorption of the therapeutic compound solution by the hydrogel coating. Other factors affecting the dosage are the concentration of the therapeutic compound in the solution applied to the coating, and the drugreleasability of the hydrogel coating, determined by, for example, the thickness of the hydrogel coating, its resiliency, porosity and the ability of the hydrogel coating to retain the therapeutic compound, e.g. electrostatic binding or pore size, or the ionic strength of the coating, e.g. changed by changing the pH. It may be advantageous to select a hydrogel coating for a particular drug, such that the therapeutic compound is not substantially released into body fluids prior to application to the site. The release of the solid/solid solution of polymer and therapeutic compound can further be controlled by varying the ratio of therapeutic compound to polymer in the multiple layers. Coating need not be solid/solid solution of polymeric and therapeutic compound, but may instead be provided from any combination of drug and polymer applied to implant. The ratio of therapeutic substance to polymer in the solution will depend on the efficacy of the polymer in securing the therapeutic substance onto the implant and the rate at which the coating is to release the therapeutic substance to the tissues. More polymer may be needed if it has a relatively poor efficacy in retaining the therapeutic substance on the implant, and more polymer may be needed in order to provide an elution matrix that limits the elution of a very soluble therapeutic substance. Therefore, a wide therapeutic substance-to-polymer rate could be appropriate, and it could range from about 10:1 to 1:100 (U.S. Pat. No. 5,776,184). Binding of the therapeutic compound may also be accomplished by electrostatic attraction of the drug to the coating or to a coating additive or a mechanical binding, for example by employing a coating having a pore size that inhibits inward flow of body fluids or outward flow of the therapeutic compound itself, which might tend to release the therapeutic compound.

Hydrogels are particularly advantageous in that the therapeutic compound is held within the hydrogen-bond matrix formed by the gel (U.S. Pat. No. 5,674,192). Examples of hydrogels are for example HYDROPLUS.RTM (U.S. Pat. No. 5,674,192), CARBOPOL.RTM (U.S. Pat. No. 5,843,089), AQUAVENE.RTM (U.S. Pat. No. 4,883,699), HYPAN.RTM (U.S. Pat. No. 4,480,642). In some cases, the hydrogel may be crosslinked prior to lining the implant, for example the hydrogel coating on a vascular or endovascular graft may be contacted with a primer dip before the hydrogel is deposited on the implant If crosslinked it forms a relatively permanent lining on the implant surface, and if left uncrosslinked it forms a relatively degradable lining on the implant surface. For example, the longevity of a crosslinked form of a given hydrogel in the stent lining, has been at least twice to that of its uncrosslinked form (U.S. Pat. No. 5,843,089). Alternatively, the hydrogel lining may be contacted with a crosslinking agent in situ (U.S. Pat. No. 5,843,089). In general, when dry, the hydrogel coating is preferably on the order of about 1 to 10 microns thick, and typically of 2 to 5 microns. Very thin hydrogel coatings, e.g., of about 0.2-0.3 microns (dry) and much thicker hydrogel coatings, e.g., more than 10 microns (dry) are also possible. Typically, the hydrogel coating thickness may swell with a factor of about 6 to 10 or more, when hydrogel is hydrated (U.S. Pat. No. 5,674,192). Usually, the polymeric carrier will be biodegradable or bioeluting (taught for example by U.S. Pat. No. 5,954,706, U.S. Pat. No. 5,914,182, U.S. Pat. No. 5,916,585, U.S. Pat. No. 5,928,916). The carrier can also be constructed to be a biodegradable substance filling the pores, and release one or more substances into the surrounding tissue by progressive dissolution of the matrix. Subsequently the pores will open. The delivered vectors may be nucleic acids encoding for therapeutic protein, e.g. a naked nucleic acid or a nucleic acid incorporated into a viral vector or liposome. By a naked nucleic acid is meant a single or double stranded DNA or RNA molecule not incorporated into a virus or liposome. Antisense oligonucleotides, which specifically bind to complementary mRNA molecules, and thereby reduce or inhibit protein expression, can also be delivered to the implant site via the hydrogel coating (U.S. Pat. No. 5,843,089). Generally, attachment of the nucleic acid to the implant can also be done in several other ways, such as by using covalent or ionic attachment techniques. Typically, covalent attachment techniques require the use of coupling agents, such as glutaraldehyde, cyanogen bromide, p-benzoquinone, succinic anhydrades, carbodiimides, diisocyanates, ethyl chloroformate, dipyridyl disulphide, epichlorohydrin, azides, among others, without excluding any other agent, but any method that uses the described methods of this invention can be used and will be recognised by a person skilled in the art Covalent coupling of a biomolecule to a surface may create undesirable cross-links between biomolecules, and thereby destroying the biological properties of the biomolecule. Also, they may create bonds amongst surface functional sites and thereby inhibit attachment. Covalent coupling of a biomolecule to a surface may also destroy the biomolecules three-dimensional structure, and thereby reducing or destroying the biological properties (U.S. Pat. No. 5,928,916). Ionic coupling techniques have the advantage of not altering the chemical composition of the attached biomolecule, and ionic coupling of biomolecules also has an advantage of releasing the biomolecule under appropriate conditions. One example is (U.S. Pat. No. 4,442,133). The current techniques for immobilisation of biomolecules by an ionic bond have been achieved by introducing positive charges on the biomaterial surface utilising quaternary ammonium salts, polymers containing tertiary and quaternary amine groups, such as TDMAC, benzalconium chloride, cetylpyrridinium chloride, benzyldimethylstearyammonium chloride, benzylcetyidimethylammonium chloride, guanidine or biguanide moiety (U.S. Pat. No. 5,928,916). When delivering the vascular implant percutaneously, a sheath member may be included to inhibit release of the drug into body fluids during placement of the catheter. For example, it can be carbowax, gelatin, polyvinyl alcohol, polyethylene oxide, polyethylene glycol, or a biodegradable or thermally degradable polymer, e.g. albumin or pluronic gel F-127 (U.S. Pat. No. 5,674,192). The particular type of attachment method when practising the methods and compositions of the invention is not important, as long as the nucleic acids released from the implant stimulates the surrounding tissue in such a way that they are activated and, in the context of in vivo embodiments, ultimately give rise to endothelialisation of the cardiovascular or tissue implant without causing adverse reactions. The methods described herein are by no means all inclusive, and further methods to suit the specific application will be apparent to the skilled person of the art.

The composition of the present invention can be provided in unit dosage form, wherein each dosage unit, e.g. solution, gel, glue, drops and aerosol, contains a predetermined amount of the composition, alone or in appropriate combination with other active agents. The term unit dosage form, as used herein, refers to physically discrete units suitable as unitary dosages for human and animal subjects, whereby each unit contains a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved, and the particular pharmacodynamnics associated with the pharmaceutical composition in the particular host.

Accordingly, the present invention also provides a method of transferring a therapeutic gene to a host, which comprises administering the vector of the present invention either systemically or, preferably as a part of composition with the implant, using the aforementioned ways of administration or alternative ways known to those skilled in the art. The effective amount of the composition is such as to produce the desired effect in a host, which can be monitored using several end-points known to those skilled in the art. Effective gene transfer of a vector to a host cell, in accordance with the present invention, can be monitored in terms of a therapeutic effect (e.g. formation of capillaries and endothelialisation of surfaces), or further by evidence of the transferred gene or expression of the gene within the host (e.g. using the polymerase chain reaction in conjunction with sequencing, Northern or Southern hybridisations, or transcription assays to detect the nucleic acid in host cells, or using immunoblot analysis, antibody-mediated detection, mRNA or protein half-life studies, or particularised assays to detect protein or polypeptide encoded by the transferred nucleic acid, or impacted in level or function due to such transfer). One such particularised assay described in the examples includes Western immunoassay for detection of proteins encoded by the EC-SOD-gene. These methods are by no means all-inclusive, and further methods to suit the specific application will be apparent to a person skilled in the art. Moreover, the effective amount of the compositions can be further approximated through analogy to compounds known to exert the desired effect (e.g., compounds traditionally employed to inhibit restenosis can provide guidance in terms of the amount of a EC-SOD nucleic acid to be administered to a host).

Furthermore, the preferred amounts of each active agent included in the compositions according to the invention, EC-SOD is preferably included from about 0.1 micrograms to 10000 micrograms (although any suitable amount can be utilised either above, i.e. greater than about 10000 micrograms, or below, i.e. less than about 0.1 micrograms), provide general guidance of the range of each component to be utilised by the practitioner upon optimising the methods of the present invention for practice in vivo. Similarly, EC-SOD plasmids are included from 0.1 to 10000 micrograms (although any suitable amount can be utilised either above, i.e. greater than about 10000 micrograms, or below, i.e. less than about 0.1 micrograms). The EC-SOD vector preferably has between 10⁷ and 10¹³ viral particles, although any suitable amount can be utilised, either more than 10¹³ or less than 10⁷. Moreover, such ranges by no means preclude use of a higher or lower amount of a component, as might be warranted in a particular application. For instance, actual dose and schedule can vary depending on whether the compositions are administered in combination with other pharmaceutical compositions, or depending on interindividual differences in pharmacokinetics, drug disposition, and metabolism. Furthermore, the amount of vector to be added per cell will likely vary with the length and stability of the gene inserted in the vector, as well as also the nature of the sequence, and is particularly a parameter which needs to be determined empirically, and it can be altered due to factors not inherent to the methods of the present invention (for instance, the cost associated with synthesis). A person skilled in the art can easily make any necessary adjustments in accordance with the exigencies of the particular situation. The amount of gene construct that is applied to the surrounding tissue or the amount of gene composition that is applied on the implant or in the tissue, will be finally determined by the attending physician or veterinarian considering various biological and medical factors. For example, one would wish to consider the particular EC-SOD and vascular implant material, patient or animal size, age, sex, diet, time of administration, as well as any further clinical factors that may affect inhibition of connective tissue formation, such as serum levels of different factors and hormones. The suitable dosage regimen will therefore be readily determinable by a person skilled in the art in light of the coming disclosure, bearing the individual circumstances in mind.

Also, for these embodiments, when one or more different vectors (i.e. each encoding one or more different therapeutic genes) are employed in the methods described herein, the contacting of cells with various components of the present invention can occur in any order or can occur simultaneously. Preferably it occurs simultaneously.

3. Connective Tissue Inhibiting Tissue

This invention provides advantageous methods for using genes to inhibit excessive connective tissue formation and improve endothelialisation. As used here surrounding tissue refers to any or all of those cells that have the capacity to ultimately inhibit, or contribute to the inhibition of, new connective tissue after tissue trauma or after device implantation. This includes various tissues in various forms, such as for example vessel wall, pleura, pericardium, peritoneum, omentum, fat and muscle.

The particular type or types of surrounding tissue, which are stimulated with the methods and compositions of the invention, are not important, as long as the cells are stimulated in such a way that they are activated, and, in the context of in vivo embodiments, ultimately give rise to inhibition of unwanted connective tissue growth, neointima formation or promotion of endothelialisation and capillarisation of the implant.

The surrounding tissue is also used to particularly refer to those cells that are located within, are in contact with, or migrate towards the implant, and which cells directly or indirectly inhibit connective tissue formation, neointima formation or stimulate the formation of endothelium and/or capillaries. As such, microvascular endothelial cells may be cells that form capillaries, that upon stimulation further inhibit connective tissue formation or attract endothelial cells, are also considered to be surrounding tissue in the context of this disclosure, as their stimulation indirectly leads to inhibition of connective tissue formation or stimulation of endothelialisation. Cells affecting connective tissue formation or endothelialisation indirectly may do so by the elaboration of various growth factors and cytokines, or by their physical interaction with other cell types. Also, cells or tissues that in their natural environment arrive at an area of active inhibition of connective tissue formation or stimulation of implant endothelialisation and vascularisation may be surrounding tissue. Surrounding tissue cells may also be cells that are attracted or recruited to such an area. Although of scientific interest, the direct or indirect mechanisms by which surrounding tissue cells inhibit connective tissue formation or stimulate endotheliaisation is not a consideration in the practising of this invention.

Surrounding tissue cells may be cells or tissues that in their natural environment arrive at an area of active connective tissue formation or vascular prosthesis, endovascular prosthesis endothelialisation, or tissue implant vascularisation. In terms of surrounding tissue, these cells may also be cells that are attracted or recruited to such an area.

According to the invention, the surrounding cells and tissues will be those cells and tissues that arrive to the tissues or surfaces of cardiovascular implants where one wishes to inhibit connective tissue formation or endothelialisation, or cells or tissues that arrive to the surface of tissue implants that one wants to vascularise.

Accordingly, in treatment embodiments there is no difficulty associated with the identification of suitable surrounding tissues to which the present therapeutic compositions, and cardiovascular and tissue implants or other prosthetic devices should be applied. All that is required in such cases is to obtain an appropriate inhibitory and stimulatory composition, as disclosed herein, and to contact the cardiovascular or tissue implant or prosthetic device with the stimulatory composition and the surrounding tissue. The nature of this biological environment is such that the appropriate cells will become activated in the absence of any further targeting or cellular identification by the practitioner.

One aspect of the invention involves to generally administer a composition to general circulation or contact surrounding tissues with a composition comprising EC-SOD protein or gene (with or without additional genes, proteins, growth factors, drugs or other biomolecules), and a cardiovascular or tissue implant or other prosthetic devices to promote expression of said gene in said cells. As outlined, cells may be contacted in vivo. This is achieved, in the most direct manner, by simply obtaining a functional EC-SOD gene construct, and applying the construct to the cells. Contacting the cells with DNA, e.g. a linear DNA molecule, or DNA in the form of a plasmid, or some other recombinant vector or artificial chromosome that contains the gene of interest under the control of a promoter, along with the appropriate termination signals, is sufficient to achieve an uptake and an expression of DNA, with no further steps necessary.

In preferred embodiments, the process of contacting the surrounding tissue with the EC-SOD composition is conducted in vivo. Again, a direct consequence of this process is that the cells take up and express the gene, and the translational or the transcriptional product stimulates the process of decreased connective tissue formation or stimulation of endothelialisation and/or capillarisation of the implant without additional steps required by the practitioner.

4. Materials Used in the Devices According to the Invention

As used herein, the following terms and words shall have the following ascribed meanings. Implantable medical device, which for brevity will be referred to as implant, device or prosthesis will refer to an object that is fabricated, at least in part, from a biomaterial, and is intended for use in contact with bodily tissues, including bodily fluids. Biomaterial shall refer to the composition of the material used to prepare a device, which provides one or more of its tissue contacting surfaces. Porosity and inflections thereof (such as pores and porous), if not specified otherwise, shall refer to a biomaterial having small channels or passages which start at an external (e.g. first major) surface of the biomaterial and extend substantially through the biomaterial to an internal (e.g., second) surface. Rigid and inflections thereof, will, in case of a nonabsorbable biomaterial, when fabricated in the form of an implantable medical device, refer to the ability to withstand the pressures encountered in the course of its use, e.g. to retain patency and pore structure in vivo. The surface shall refer to the interface between the biomaterial and its environment. The term is intended to include the use of the word in both its macroscopic sense (e.g. the two major faces of a sheet of biomaterial), as well as in its microscopic sense (e.g. the lining of pores traversing the material). The term “attach” and its derivatives refer to adsorption, such as physisorption, or chemisorption, ligand/receptor interaction, covalent bonding, hydrogen bonding, or ionic bonding of a polymeric substance or nucleic acids to the implant.

The type of cardiovascular, tissue implants and other prosthetic devices that may be used in the compositions, devices and methods of the invention is virtually limitless, as long as they are tissue compatible. Thus, devices of the present invention include medical devices intended for prolonged contact with blood, bodily fluids or tissues, and in particular, those that can benefit from inhibition of unwanted or excessive connective tissue growth and fibrosis or stimulation of the capillary endothelialisation when used for in vivo applications. Preferred devices are implantable in the body, and include cardiovascular implants, tissue implants, artificial organs, such as the pancreas, liver, and kidney, and organ implants, such as breast, penis, skin, nose, ear and orthopedic implants. The significance of inhibition of connective tissue formation or capillary endothelialisation will vary with each particular device, depending on the type and purpose of the device. The inhibition of connective tissue formation protects the device from excessive scar tissue formation, strictures and fibrosis. Ingrown capillaries can provide endothelial cells to line surfaces of vascular implants, protect tissue implants from infection, carry nutrients to the cells in the device and make it possible for sensors to sense substance levels in circulation. This means that the implant has all the features commonly associated with biocompatibility, in that they are in a form that does not produce an adverse, an allergic, or any other untoward reaction when administered to a mammal. They are also suitable for being placed in contact with the tissue surrounding the implant. The latter requirement takes factors, such as the capacity of the said implants to provide a structure for the developing vascular endothelium or to resist unwanted connective tissue formation, into consideration.

Preferred biomaterial are those that provide sufficient rigidity for their intended purposes in vivo. For use in forming a vascular graft and cardiovascular patch, for instance, the biomaterial will be of sufficient rigidity to allow the graft to retain graft patency in the course of its intended use. The choice of implant material will differ according to the particular circumstances and the site where the vascular or tissue implant is implanted. Vascular prosthesises are made of biomaterials, selected from the group consisting of e.g. tetrafluoroethylene polymers, aromatic/aliphatic polyester resins, polyurethans, and silicone rubbers. However, any type of biocompatible microporous mesh may be used. The said biomaterials can be combined with each other or other substances, such as polyglycolic acid, polylactic acid, polydioxone and polyglyconate. Preferred are expanded polytetrafluorethylene and Dacron. Dacron may be with or without velour, or modified in some other way. Dacron is usually woven, braided or knitted and suitable yarns are between 10 and 400 deniers. The nodal regions of ePTFE are composed of nonporous PTFE that serves to provide tear resistance (e.g. for sutures and resistance to aneurysmal dilatation). The internodal regions are composed of fibres of PTFE, which serve to connect the nodes with the spaces between the fibres providing the porosity referred to herein. The nodal size can be expressed as the percentage of the tissue-contacting surface that is composed of nodal PTFE. The distance between nodes can be expressed as the average fibril length. In turn, the porosity is commonly expressed as the internodal distance (i.e. the average distance from the middle of one node to the middle of the adjacent node). Preferred ePTFE materials have nodes of sufficient size and frequency to provide adequate strength (e.g., with respect to aneurysmal dilatation) and internodal regions of sufficient frequency and fibre length to provide adequate porosity (to allow for capillary endothelialisation). Given the present specification, those skilled in the art will be able to identify and fabricate devices using biomaterials having a suitable combination of porosity and rigidity. Biomaterials are preferably porous to allow the attachment and migration of cells, which may be followed by the formation and growth of capillaries into the surface. Suitable pores can exist in the form of small channels or passages, which start at an external surface and extend partially or completely through the biomaterial. In such cases, the cross sectional dimensions of the pore capillary diameter are greater than 5 microns and typically less than 1 mm. The upper pore size value is not critical as long as the biomaterial retains sufficient rigidity, however it is unlikely that useful devices would have a pore size greater than about 1 mm. Such pore dimensions can be quantified in microscope. As will be understood by those skilled in the art, several modifications of the graft materials and surfaces can be made, such as precoating with, for example, proteins (see e.g. U.S. Pat. Nos. 5,037,377, 4,319,363), non-heparinised whole blood and platelet rich plasma, glow-discharge modifications of surfaces, adding pluronic gel, fibrin glue, fibronectin, adhesion molecules, covalent bonding, influencing surface charges, with for example carbon (U.S. Pat. Nos. 5,827,327, 4,164,045), and treating with a surfactant or cleaning agent, without excluding any other method. Moreover, the implant can be constructed as a hybrid of different internodal distances for the inner and outer surfaces, such as 60 microns as an outer value and 20 microns as an inner value, for the internodal distances (HYBRID PTFE). Also, more layers with different internodal distances may be used. They are all intended to fall within the scope of the present invention when not inhibiting endothelialisation. Potential biodegradable vascular implants may be used in connection with the compositions, devices and methods of this invention. For example, biodegradable and chemically defined polylactic acid, polyglycolic acid, matrices of purified proteins, semi-purified extracellular matrix compositions and also collagen can be employed. Also, naturally occuring autogenic, allogenic and xenogenic material, such as an umbilical vein, saphenous vein, native bovine artery or intestinal sub-mucosal tissue may be used as a vascular or other implant material. Examples of clinically used grafts are disclosed in U.S. Pat. No. 4,187,390, U.S. Pat. No. 5,474,824 and U.S. Pat. No. 5,827,327. Biodegradable or bioabsorbable materials, such as homopolymers e.g. poly-paradioxanone, polylysine or polyglycolic acid and copolymers; e.g., polylactic acid and polyglycolic acids or other bio materials, may be used either alone or in combination with other materials as the vascular graft or other implant material, as long as they provide the required rigidity. Also, other biological materials, such as intestinal submucosa, matrices of purified proteins and semi-purified extracellular matrix compositions may be used. Appropriate vascular grafts or other prosthetic implants will both deliver the gene composition and also provide a surface for new endothelium growth, i.e., will act as an in situ scaffolding through which endothelial cells may migrate. It will be understood by a person skilled in the art that any material with biocompatibility, rigidity will be acceptable to be used with the invention. It will also be understood that inhibition of excessive connective tissue formation can occur in connection of an autologous vessel to autologous vessel, allogene vessel to autologous vessel, autologous vessel to synthetic vessel or any type of vessel to another vessel either end-to-end, end-to-side, side-to-side or combination of multiple side-to-side and end-to-side-anastomosis as long as there is a connection between the vessels with an anastomotic area. Also the hyperplasia can be inhibited in any part of the graft.

Background for cardiovascular patches is well described in for example U.S. Pat. No. 5,104,400, U.S. Pat. No. 4,164,045, U.S. Pat. No. 5,037,377. In the case of vascular patches, one side of the patch engages the blood while the other side engages other surrounding tissues to promote transgraft growth of the endothelial cells. In the case of intracardiac patches, blood engages both sides of the patch. Preferred biomaterials are those that provide sufficient rigidity in vivo. A vascular patch biomaterial will be of sufficient rigidity to allow the patch to retain its form and pore-structure in the course of its intended use. The choice of patch material will differ according to the particular circumstances and site where the vascular patch is implanted. Vascular patch is made of synthetic biomaterial, such materials include, but are not limited to, tetrafluoroethylene polymers, aromatic/aliphatic polyester resins, polyurethans, and silicone rubbers, however any type of biocompatible microporous mesh may be used. The said biomaterials can be combined with each other or other substances such as polyglycolic acid. Preferred are expanded polytetrafluorethylene and Dacron. Dacron is usually woven, braided or knitted, and with or without velour, and suitable yarns are between 10 and 400 deniers. The nodal regions of ePTE are composed of nonporous PTFE that serves to provide tear resistance (e.g. for sutures and resistance to aneurysmal dilatation). The internodal regions are composed of fibres of PTFE, which serve to connect the nodes, with the spaces between the fibres providing the porosity referred to herein. The nodal size can be expressed as the percentage of the tissue-contacting surface that is composed of nodal PTFE. The distance between nodes can be expressed as the average fibril length. In turn the porosity is commonly expressed as the internodal distance (i.e. the average distance from the middle of one node to the middle of adjacent node). Preferred ePTFE materials have nodes of sufficient size and frequency to provide adequate strength (e.g., with respect to aneurysmal dilatation) and internodal regions of sufficient frequency and fibre length to provide adequate porosity (to allow for capillary endothelialisation). Such materials will provide fewer though thicker nodes, which will in turn confer significantly greater strength in vivo. Given the present specification, those skilled in the art will be able to identify and fabricate devices using biomaterials having a suitable combination of porosity and rigidity. Biomaterials are preferably porous to allow the attachment and migration of cells, which may be followed by the formation and growth of capillaries into the luminal surface. Suitable pores can exist in the form of small channels or passages, which start at an external surface and extend through the biomaterial. In such cases, the cross sectional dimensions of the pores are larger than the diameter of a capillary 5 microns and are typically less than 1 mm. Upper pore size value is not critical as long as the biomaterial retains sufficient rigidity. However, it is unlikely that useful devices would have pore size greater than about 1 mm. Such pore dimensions can be quantified in microscope. As will be understood by a person skilled in the art, several modifications of graft materials and surfaces can be made, such as precoating with for example proteins (for example, U.S. Pat. No. 5,037,377, U.S. Pat. No. 4,319,363), non-heparinised whole blood and platelet rich plasma, glow-discharge modifications of surfaces, adding poloxemers, fibrin glue, adhesion molecules, covalent bonding, influencing surface charges with for example carbon (U.S. Pat. No. 5,827,327, U.S. Pat. No. 4,164,045), treating with a surfactant or cleaning agent, without excluding any other method. Also the implant can be constructed as a hybrid of different internodal distances in inner and outer surface, such as outer 60 microns and inner 20 microns in internodal distance (HYBRID PTFE). Even more layers with different internodal distances may be used. They all are intended to fall in the scope of present invention when not inhibiting endothelialisation. Potential biodegradable materials may be used in connection with the compositions, devices and methods of this invention, for example homopolymers e.g. poly-paradioxanone, polylysine or polyglycolic acid and copolymers e.g., polylactic acid and polyglycolic acids or other bio materials, such is as matrices of purified proteins and semi-purified extracellular matrix compositions may be used either alone or in combination with other materials as cardiovascular patch material, as long as they provide the required rigidity. Naturally occurring autogenic, allogenic and xenogenic material such as an umbilical vein, saphenous vein, native bovine artery, pericardium or intestinal submucosal tissue may also be used as cardiovascular patch material. Examples of clinically used vascular patches are disclosed in U.S. Pat. No. 5,037,377, U.S. Pat. No. 5,456,711, U.S. Pat. No. 5,104,400, U.S. Pat. No. 4,164,045. Appropriate vascular patches will both deliver the gene composition and also provide a surface for new endothelium growth, i.e., will act as an in Situ scaffolding on which and through which endothelial cells may migrate. Preferably, nucleic acids are attached to the side engaging the tissues surrounding the vessel. Appropriate intracardiac patches will both deliver the gene composition to the surrounding tissues and provide a surface for new endothelium growth, i.e., will act as an in situ scaffolding on which and through which endothelial cells may migrate. Preferably, nucleic acids are attached to both intracardiac patch surfaces. Alternatively, nucleic acids may be attached to one of the intracardiac patch surfaces. It will be understood by a person skilled in the art, that any material with biocompatibility and rigidity will be acceptable to be used with the invention.

Stent herein means a medical implant in the form of a hollow cylinder, which will provide support for the body lumen when it is implanted in contact with a site in the wall of a lumen to be treated. They can be of several different designs such as tubular, conical or bifurcated. The configuration can be such as a coiled spring, braided filament, perforated tube, slit tube, and zigzag, or any other variant. Preferably, in the case of vascular stents it is adapted for use in blood vessels in a way that the stent has an outer, lumen-contacting surface, and an inner, blood-contacting surface. Many stents of the art are formed of individual member(s), such as wire, plastic, metal strips, or mesh, which are bent, woven, interlaced or otherwise fabricated into a generally cylindrical configuration. The stent can also have underlying polymeric or metallic structural elements, onto which elements, a film, is applied (U.S. Pat. No. 5,951,586). Stents have been classified into either self-expanding or pressure expandable. The terms expand, expanding, and expandable are used herein to refer to diametrically adjustable intraluminal stents. When the self expanding stents are positioned at the treatment site with a delivery catheter, they are supposed to radially expand to a larger diameter after being released from a constraining force, which force restricts them to a smaller diameter and conform a surface contact with a blood vessel wall or other tissue without exertion of outwardly directed radial force upon stent. Stents of this type include stents of braided or formed wire. Self-expanding stents may also expand to a size as defined by thermal memory. The pressure-expandable stents are fabricated of malleable or plastically deformable material, typically formed of metal wire or metal strips. The collapsed stent is taken to the treatment site with a delivery catheter, and is then radially expanded with a balloon or other stent-expansion apparatus to its intended operative diameter. Thread elements or strands formed of metal are generally favoured, for applications requiring flexibility and effective resistance to radial compression after implantation. The favourable combination of strength and flexibility is largely due to the properties of the strands after they have been age hardened, or otherwise thermally treated in the case of polymeric strands. The braiding angle of the helical strands and the axial spacing between adjacent strands also contribute to strength and flexibility.

Stent wires may be of metal, inorganic fibres, ceramic or organic polymers. They should be elastic, strong, biocompatible, and fatigue and corrosion resistant. For example, core wires made of metals, such as stainless steel or gold or other relatively pliable non-toxic metals and alloys that do not degrade during the time of implantation or are not subject to severe degradation (corrosion) under the influence of an electric current, are usually chosen. Such metals include, but are not limited to, platinum, platinum-iridium alloys, copper alloys, with tin or titanium, nickel-chrome-cobalt alloys, cobalt based alloys, molybdenium alloys, nickel-titanium alloys. The strands need not be of metal and may for example be of a polymeric material such as PET, polypropylene, PEEK, HDPE, polysulfone, acetyl, PTFE, PEP, and polyurethane without excluding any other substance (other variants: polytetrafluorethylene, fluorinated ethylene propylene, polytetrafluorethylene-perfluoroalkyl vinyl ether copolymer, polyvinyl chloride, polypropylene, polyethylene terephthalate, broad fluoride and other biocompatible plastics). Also, a biodegradable or bioabsorbable rnaterial, such as homopolymers e.g. poly-paradioxanone, polylysine or polyglycolic acid and copolymers, e.g. polylactic acid and polyglycolic acids, polyurethane, or other biomaterials, may be used either alone or in combination with other materials as the stent material. Such monofilament strands range from 0.002 to 0.015 inches in diameter but of course the diameter could vary depending on the lumen size and the degree of support needed. Also antithrombotic, anti-platelet, vasodilative, antiproliferative, antimigratory, antifibrotic, anti-inflammatory agents and more specifically, heparin, hirudin, hirulog, etritinate, freskolin, rapamycin, sirolimus, paclitaxel, tacrolimus, dexamethasone, cytochalasine D and Actinomycin C and the like, may be attached the stent. Examples of clinically used stents are disclosed in U.S. Pat. No. 4,733,665, U.S. Pat. No. 4,800,882, U.S. Pat. No. 4,886,062 incorporated here by reference.

Stent grafts, also called covered stents, for transluminal implantations include a resilient tubular interbraided latticework of metal or polymeric monofilaments, a tubular interbraided sleeve formed of a plurality of interwoven textile strands, and an attachment component that fixes the latticework and the sleeve together, in a selected axial alignment with one another, engaged with one another and with a selected one of the latticework and the sleeve surrounding the other, whereby the latticework structurally supports the sleeve. It is ensured that the latticework and the sleeve behave according to substantially the same relationship governing the amount of radial reduction that accompanies a given axial elongation. The sleeve may be exterior or interior to the latticework, or the latticework may be integrated in the sleeve, and it can be continuous or discontinuous. Several prosthesis constructions have been suggested for composite braided structures that combine different types of strands, e.g. multifilament yarns, monofilaments, fusible, materials and collagens. Examples are found in WO91/10766. Textile strands are preferably multifilament yarns, even though they can be monofilaments. In either case the textile strands are much finer than the structural strands, ranging from about 10 denier to 400 denier. Individual filaments of the multifilament yams can range from about 0.25 to about 10 denier. Multifilament yarns can be composed of various materials, such as PET, polypropylen, polyethylen, polyurethane, HDPE, silicone, PTFE, polyolefins and EPTFE. By modifying the yarns it is possible to modify sleeve qualities, for example untwisted flat filaments provide thinner walls, smaller intersticies between yarns so achieving lower permeability, and higher yarn cross-section porosity for capillary transgraft growth. Porous expanded PTFE film has a microstructure of nodes interconnected by fibrils and may be made as taught by for example U.S. Pat. Nos. 3,953,566, 4,187,390 and 4,482,516. Suitable pores can exist in the form of small channels or passages starting at an external surface and extending through the biomaterial. In such cases the cross-sectional dimensions of the pores are larger than the diameter of a capillary 5 microns, and are typically less than 1 mm. Upper pore size value is not critical so long as the biomaterial retains sufficient rigidity, however it is unlikely that useful devices would have pore size greater than about 1 mm. Such pore dimensions can be quantified in microscope. As will be understood by those in the art several modifications of stent graft materials and surfaces can be made such as precoating with proteins, non-heparinised whole blood and platelet rich plasma, glow-discharge modifications of surfaces, adding pluronic gel, fibronectin, fibrin glue, adhesion molecules, covalent bonding, influencing surface charges with for example carbon (U.S. Pat. No. 5,827,327, U.S. Pat. No. 4,164,045), treating with a surfactant or cleaning agent, mechanically changing the characteristics, such as drilling holes, adding grooves and changing the end angles without excluding any other method. Also the implant can be constructed as a hybrid of different internodal distances in inner and outer surface such as outer 60 microns and inner 20 microns in internodal distance (HYBRID PTFE). Even more layers with different internodal distances can be used. They all are intended to fall in the scope of present invention when not promoting unwanted connective tissue growth or inhibiting endothelialisation. The fibrils can be uni-axially oriented, that is oriented in primarily one direction, or multiaxially oriented, that is oriented in more than one direction. The term expanded is used herein to refer to porous expanded PTFE. It will be understood by a person skilled in the art, that any material with biocompatibility will be acceptable to be used with the invention. Examples of clinically used stent grafts are disclosed in U.S. Pat. No. 5,957,974, U.S. Pat. No. 5,928,279, U.S. Pat. No. 5,925,075, and U.S. Pat. No. 5,916,264.

Also, naturally occuring autologous, allogenic or xenogenic materials, such as arteries, veins and intestinal submucosal can be used in stent grafts, such as an umbilical vein, saphenous vein, or native bovine artery. Potential biodegradable vascular implants may be used as stent grafts in connection with the compositions, devices and methods of this invention, for example biodegradable and chemically defined polylactic acid, polyglycolic acid, matrices of purified proteins, semi-purified extracellular matrix compositions. Appropriate vascular grafts and stent grafts will both deliver the gene composition and also provide a surface for new endothelium growth, i.e., will act as an in situ scaffolding through which endothelial cells may migrate and preferably inhibit unwanted tissue growth or restenosis. The particular design of the implants that are implanted using the methods and compositions of the invention are not important, as long as connective tissue formation can be inhibited and they act as scaffolds through which endothelium can migrate, in the context of in vivo embodiments, and ultimately give rise to endothelialisation of the implant.

A variety of catheter systems are useful for delivering the interventional stents and stent grafts into the desired site. The chosen type is not important as long as the methods of present invention are used.

Heart valves are well known in the art and operate hemodynamically as a result of the pumping action of the heart Generally, there is an annular body having an interior surface, which defines a blood flow passageway, and which has one or multiple occluders sup ported thereon, for alternately blocking, and then allowing the blood flow in a predetermined direction. Heart valve prostheses are of various different designs, and of autologous, allogenic, xenogenic or synthetic material. The mechanical valve annular housing, also called annular body, and the valving members, can be made of any biocompatible and nonthrombogenic material, that also will take the wear they will be subjected to. There are various different designs, such as a circular valve housing and a valving member, such as a spherical member or ball, pivoting disc, poppet disc, and leaflet members, such as single or multiple leaflet constructs, for example two flat leaflets, leaflets with conical, semiconical and cylindrical surfaces. The orifice ring can be made of various materials, such as a pyrocarbon coated surface, a silver coated surface or from solid pyrolytic carbon (described in U.S. Pat. No. 4,443,894), and leaflets may be made of one substrate, such as polycristalline graphite, plastic, metal or any other rigid material, and then coated with another, such as pyrolytic carbon (e.g. in U.S. Pat. No. 3,546,711, and U.S. Pat. No. 3,579,645). Circular valve housing can be porous, (here referred as having a porous surface and a network of interconnected interstitial pores below the surface in fluid flow communication with the pores, see U.S. Pat. No. 4,936,317), or nonporous, and suitable means, such as peripheral groove or a pair of flats can be provided for attaching a suturing ring to the annular body to facilitate sewing or suturing of the heart valve to the heart tissue. The suturing member may have a rigid annular member or sleeve surrounding the base. The sleeve may be of a rigid material, such as metal, plastic or alike. The sleeve may have collars of fabric, such as Teflon or Dacron (RE31,400). The valve may have further members, such as a cushioning member and a shock-absorbing member. Examples of mechanical heart valves are described in U.S. Pat. No. 3,546,711, U.S. Pat. No. 4,011,601, U.S. Pat. No. 4,425,670, U.S. Pat. No. 3,824,629, U.S. Pat. No. 4,725,275, U.S. Pat. No. 4,078,268, U.S. Pat. No. 4,159,543, U.S. Pat. No. 4,535,484, U.S. Pat. No. 4,692,165, U.S. Pat. No. 5,035,709, and U.S. Pat. No. 5,037,434.

Xenografts, allografts or autografts can be used as tissue valves. When an autologous graft is used, usually the pulmonary valve is operated to the aortic position—a Ross operation. Allografts, also called homografts, are of cadaveric origin. Xenograft bioprosthetic heart valves are usually of porcine origin. They can be stented or stentless. The traditional stented valves may be designed to have a valving element, stent assembly and a suture ring. The stent may be cloth covered. All the known stent materials can be used in the stent, including but not limited to titanium, Dehin, polyacetal, polypropylene, and Elgiloy. As is known by a person skilled in the art, there are several ways to manipulate tissue valves. For example a bioprosthesis may be made acellular (Wilson, Ann. Thorac Surg., 1995;60 (2 suppl): S353-8) or preserved in various ways, such as with glutaraldehyde, glycerol (Hoffman), dye-mediated photooxidation (Schoen, J. Heart Valve Dis., 1998; 7(2):174-9), and if preserved with glutaraldehyde, glutaraldehyde can be neutralised by aminoreagents (e.g. U.S. Pat. No. 4,405,327). Homografts can be deendothelialised. Examples of tissue heart valves are described in U.S. Pat. No. 3,755,823, U.S. Pat. No. 4,441,216, U.S. Pat. No. 4,172,295, U.S. Pat. No. 4,192,020, U.S. Pat. No. 4,106,129, U.S. Pat. No. 4,501,030, and U.S. Pat. No. 4,648,881. Also, there exists an extensive scientific literature in the subject. It will be understood by a person skilled in the art, that any material or tissue with biocompatibility to inhibit unwanted connective tissue growth or allow endothelial growth will be acceptable. Genes can be attached to the heart valve prostheses by various methods but the method is not important as long as gene is taken up by the surrounding tissue and EC-SOD is produced and excessive connective tissue formation and fibrosis inhibited or angiogenesis is stimulated, which results in endothelialisation of the orifice ring and/or the valving member surface. Nucleic acids or a composition comprising nucleic acids may be attached to whole or parts of the heart valves. Preferably, in tissue valves nucleic acids will be attached to the whole surface and stent assembly, and in mechanical valves to annular body and sewing ring.

Tissue implants can be made of various materials, such as polyethylene, polypropylene, polytetrafluorethylene (PTFE), cellulose acetate, cellulose nitrate, polycarbonate, polyester, nylon, polysulfone, mixed esters of cellulose, polyvinylidene difluoride, silicone, collagen and polyacrylonitrile. Preferred support materials for tissue engineering are synthetic polymers, including oligomers, homopolymers, and copolymers resulting from either addition or condensation polymerisations. Examples of tissue implants are described in e.g. U.S. Pat. No. 5,314,471, U.S. Pat. No. 5,882,354, U.S. Pat. No. 5,874,099, U.S. Pat. No. 5,776,747, and U.S. Pat. No. 5,855,613. It will be understood by the person skilled in the art that any material with biocompatibility to allow endothelial growth and/or capillarisation will be acceptable. EC-SOD genes can be attached to the implant by various methods, but the method is not important as long as gene is taken up by the surrounding tissue and EC-SOD is produced and fibrosis is inhibited or angiogenesis is stimulated resulting in endothelialisation and/or capillarisation of the implant.

Background for anastomotic devices also called graft connectors is well described in U.S. Pat. No. 5,904,697 and U.S. Pat. No. 5,868,763. Generally, anastomotic devices are employed either in end-to-end anastomosis or end-to-side anastomosis. This invention comprises end-to-side anastomotic devices, preferably those anastomotic devices that have an anchoring member being implanted intraluminally to the target vessel and exposed to blood, such as SOLEM GraftConnector™. The term “anchoring member” is here referred to the member forming the attachment with the target vessel. The term “coupling member” or “connecting member” here refers to the member that forms attachment with the bypass graft vessel. Anchoring member and coupling member may form one single unit or be separated being connected during the procedure. Additional members such as a handle and pins may be comprised. The intraluminal anchoring member may be of various design, preferably it is a tubular structure. The intraluminal anchoring member may be made of any biocompatible material such as metal, ceramic, plastic, polymer, PTFE, DACRON, PET, polypropylen, polyethylen, polyurethane, HDPE, silicone, polyolefins and ePTFE or combination of several structures. Also a biodegradable or bioabsorbable material such as homopolymers e.g. poly-paradioxanone, polylysine or polyglycolic acid and copolymers e.g. polylactic acid and polyglycolic acids or other bio materials may be used either alone or in combination with other materials. Anastomotic device may be porous, partly porous, or nonporous. Preferably the connecting member is nonporous and the anchoring member is porous. Alternatively both the connecting member and anchoring member are porous. If porous, the cross sectional dimensions of the pore capillary diameter are greater than 5 microns and typically less than 1 mm. Upper pore size value is not critical so long as the biomaterial retains sufficient rigidity, however it is unlikely that useful devices would have pore size greater than about 1 mm. Such pore dimensions can be quantified in microscope. Suitable pores can exist in the form of channels or passages starting at the external surface and extend through the biomaterial. As will be understood by those in the art several modifications of graft connector design materials and surfaces can be made such as precoating with proteins, non-heparinised whole blood and platelet rich plasma, glow-discharge modifications of surfaces, adding pluronic gel, fibrin glue, adhesion molecules, covalent bonding, influencing surface charges with for example carbon (U.S. Pat. No. 5,827,327, and U.S. Pat. No. 4,164,045), treating with a surfactant or cleaning agent, mechanically changing the surface characteristics, such as adding grooves and changing the end angles without excluding any other method. Also the implant can be constructed as a hybrid of different internodal distances in inner and outer surface, such as outer 60 microns and inner 20 microns in internodal distance (such as HYBRID PTFE). Even more layers with different internodal distances may be used. They all are intended to fall in the scope of present invention when not inhibiting endothelialisation. Genes can be attached to the implant by various methods, but the method is not important as long as the gene is taken up by the surrounding tissue and EC-SOD produced, connective tissue formation inhibited and angiogenesis is stimulated resulting in endothelialisation and/or capillarisation of the implant. Appropriate graft connectors will both deliver the gene composition and also provide a surface for new endothelium growth, i.e., will act as an in situ scaffolding through which endothelial cells may migrate. It will be understood by a person skilled in the art that any material with biocompatibility, rigidity and porosity to allow transgraft growth will be acceptable.

Suture materials are well known in the art. “Filament” is here referred a single, long, thin flexible structure of a non-absorbable or absorbable material. It may be continuos or staple. “Absorbable” filament is here referred one, which is absorbed, that is digested or dissolved, in mammalian tissue. Sutures may be monofilament i.e. single filament strands or multifilament i.e. several strands in a braided, twisted or other multifilament construction and are made of wide variety of materials both natural, such as metal, silk linen, cotton and catgut, and synthetic, such as nylon, polypropylene, polyester, polyethylene, polyurethane, polylactide, polyglycolide, copolymers of lactide and glycolide. Sutures may be porous (U.S. Pat. No. 4,905,367, U.S. Pat. No. 4,281,669) or nonporous and they can be coated with various materials described in for example in U.S. Pat. No. 4,185,637, U.S. Pat. No. 4,649,920, U.S. Pat. No. 4,201,216, U.S. Pat. No. 4,983,180, and U.S. Pat. No. 4,711,241 or uncoated. Nucleic acids may be attached to any suture material to inhibit unwanted connective tissue growth or promote endothelialisation of sutures. Attachment of the nucleic acids is particularly useful with synthetic non-absorbable vascular sutures. If multifilament suture is to be coated, it is not necessary that every filament within the suture be individually or completely coated. Sizes of suture materials usually range between 12-0 U.S.P. size 0.001 mm to size 2 U.S.P. with outer diameter 0.599 mm. Suture materials may be with or without needle in one or both ends and needle may be attached to the suture material by any of the methods known in the art, such as by defining a blind hole, i.e. a cylindrical recess, extending from a proximal end face of the suture needle along the axis thereof. The length of the suture-mounting portion is generally equal to or slightly greater than the length of the hole. A suture is inserted into the hole and then the suture-mounting portion is crimped, i.e. deformed or compressed, to hold the suture. Alternatively, the suture may be secured by addition of cement material to such blind hole (for example in U.S. Pat. No. 1,558,037). Also adhesive and bonding agents may be used, such as in U.S. Pat. No. 2,928,395, U.S. Pat. No. 3,394,704. Also other modifications may be employed such as in U.S. Pat. No. 4,910,377, U.S. Pat. No. 4,901,722, U.S. Pat. No. 4,890,614, U.S. Pat. No. 4,805,292, and U.S. Pat. No. 5,102,418. The surgical needle itself may be made of various materials, such as medically acceptable stainless steel of required diameter. The suture attachment to the needle may be standard i.e. the suture is securely attached and is not intended to be separable therefrom, except by cutting or severing the suture, or detachable or removable i.e. be separable in response to a force exerted by the surgeon (U.S. Pat. No. 3,890,975, U.S. Pat. No. 3,980,177, and U.S. Pat. No. 5,102,418). Surgical needles may be of various form such as ¼ circle, ⅜ circle, ½ curve, ½ circle, ⅝ circle, or straight and the needle distal point may be taper point, taper cut, reverse cutting, precision point, spatula-type, and the like. The amount of nucleic acid attached to the suture material or to the composition coating the suture will vary depending upon the construction of the fibre, e.g. the number of filaments and tightness of braid or twist and the composition, solid or solution applied. It will be understood by the skilled person that any material with biocompatibility to allow inhibition of connective tissue hyperplasia will be acceptable. Genes can be attached to the sutures by any of the methods described in this disclosure or any other method if so preferred. After gene has been taken up by the surrounding tissue, EC-SOD is produced and excessive connective tissue growth is inhibited and endothelialisation stimulated resulting in endothelialisation of the suture material surface.

Surgical pledgets are well known in the art. It will be understood by a person skilled in the art, that any material with biocompatibility to allow endothelial growth will be acceptable. Genes can be attached to the surgical pledgets by any method included in this disclosure, or any other method After gene is taken up by the surrounding tissue, EC-SOD is produced and excessive connective tissue growth inhibited and angiogenesis is stimulated resulting in endothelialisation of the implant.

Physical and chemical characteristics, such as e.g. biocompatibility, biodegradability, strength, rigidity, porosity, interface properties, durability and even cosmetic appearance may be considered in choosing the said vascular or tissue implant, as is well known for those skilled in the art. Also, an important aspect of the present invention is its use in connection with other implants having the advantage of avoidance of excessive connective or fibromuscular tissue growth or vascularisation of the interface with the tissues, including implants themselves and functional parts of the implant, such as tissue chambers, pacemaker wires, indwelling vascular catheters for long time use and the like. The surface may be coated or pores filled with nucleic acids or with a material having an affinity for nucleic acids, and then the coated-surface may be further coated with the gene or nucleic acid that one wishes to transfer. The available chemical groups of the adsorptive, may be readily manipulated to control its affinity for nucleic acids, as is known to those skilled in the art.

Experimental Section

### EXAMPLE

EC-SOD Expression Plasmid

A rabbit lung cDNA library (Clontech # TL1010a) was screened by plaque hybridization using a partial rabbit EC-SOD cDNA (genbank X78139; EC-SOD encoding bases 126-465) as a probe (Hiltunen et al., 1995, Hiltunen, T., Luoma, J., Nilkari, T. and Ylä-Herttuala, S.: Induction of 15-lipoxygenase mRNA and protein in early atherosclerotic lesions. Circulation 92 (1995) 3297-3303). Positive clones were purified by a standard method (Ausubel, F. M., Brent, R., Kingston, R. E., Moore, D. D., Seidman, J. G., Smith, A. J. and Struhl, K. (eds.): Current protocols in molecular biology. John Wiley & Sons, Inc., USA, 1995) and were found to contain the 3′ regions of the EC-SOD cDNA. The 5′ end of the coding sequence was amplified from rabbit genomic DNA by PCR using primers specific to EC-SOD gene (genbank AJ007044); 5′-GAT GCT GGC GTT GGT GTG CTC-3′/5′-GCA CGG CCA GCG GGT TGT AGT-3′. The 5′ and 3′ fragments of the cDNA were subsequently ligated to produce the entire open reading frame of EC-SOD gene which was further subcloned into pHHT631 expression vector (Mizushima, S. and Nagata, S.: pEF-BOS, a powerful mammalian expression vector. Nucleic. Acids. Res. 18 (1990) 5322) under elongation factor 1α promoter (pEC-SOD1α). DNA sequencing was done using ALF automated DNA sequencer (Pharmacia), and the sequence analyses were performed with the GCG program package (Devereux, J., Haeberli, P. and Smithies, O.: A comprehensive set of sequence analysis programs for the VAX. Nucleic. Acids. Res. 12 (1984) 387-395). The expression cassette of pEC-SOD 1α was further cloned into an adenovirus vector (AdBgIII) for adenovirus construction as described previously (Kozarsky, K. F. and Wilson, J. M.: Gene therapy: adenovirus vectors. Curr. Opin. Genet. Dev. 3 (1993) 499-503).

EC-SOD Activity Analysis

The efficiency of adenovirus gene transfer was determined by measuring total SOD activity from the plasma as described in the literature (Marklund S: Spectrophotometric study of spontaneous disproportionation of superoxide anion radical and sensitive direct assay for superoxide dismutase. J. Biol. Chem. 1976; 251:7504-7507). Briefly, a 25-50 μl plasma sample was added to 3 ml 50 mM AMP/HCl pH 9.5/0.2 MM DTPA buffer and blanked before addition of kaliumperoxide (KO₂) substrate in 50 mM NaOH/0.5 mM DTPA (Sigma). The reaction was followed for five minutes at 250 nm (Lambda Bio, Perkin Elmer). The activity was calculated by determining the half-times of O₂⁻ decay at A₂₅₀. One unit in the assay is defined as the activity that brings about a decay in O₂⁻ concentration at a rate of 0.1 s⁻¹ in 3 ml buffer and corresponds to 8.6 ng of human EC-SOD as quantified by ELISA (Marklund S: Spectrophotometric study of spontaneous disproportionation of superoxide anion radical and sensitive direct assay for superoxide dismutase. J. Biol. Chem. 1976; 251:7504-7507).

RT-PCR Analysis

The expression of EC-SOD messenger RNA was studied using Enhanced avian RT-PCR kit (Sigma). Total RNA for the analysis was isolated using Trizol reagent (Gibco BRL), and contaminating DNA was removed by incubating RNA prep 15 minutes at 37° C. with DNAse (Promega). The reactions were as follows RT: 80° C. for 10 minutes, 25° C. for 15 minutes, and 60° C. for 50 minutes. 10 μl aliquots were used for following PCR reaction with primers 5′-GGATGTTGCAAGTGACCAGGC-3′ and B 5′-GCACGGCCAG-CGGGTTGT-AGT-3′. Reaction cycle was started with a 5 minute denaturation step at 96° C. followed by 29 additional cycles; 96° C. for 1 minute, 60° C. for 1 minute, and 70° C. for 1 minute. The reaction was finished by 10 minutes incubation at 72° C.

Expression of LacZ and EC-SOD

Biodistribution of adenovirus was determined by X-gal staining from LacZ group and by RT-PCR from EC-SOD group. X-gal staining was frequently seen in the spleen, lung and liver but not in the other tissues. RT-PCR analysis for EC-SOD expression showed similar pattern of tissue distribution as LacZ staining suggesting that in addition to vascular wall adenovirus also transduces other tissues. Total plasma SOD activity, which was measured before the gene transfer and 3, 7, and 14 days after the gene transfer was shown to be significantly (p<0.01) lower three days after the gene transfer in the control rabbits as compared to the EC-SOD group showing that adenovirus mediated EC-SOD gene transfer prevented reduction in total plasma SOD activity.

Histological Analysis

The effect of adenovirus mediated gene transfer of EC-SOD on intimal thickening was evaluated in a rabbit restenosis model. Briefly, 28 New Zealand White rabbits were kept on 0.25% cholesterol rich diet for two weeks before balloon catheter mediated denudation of aortic endothelium. The animals were anaesthetised with s.c. 0.5 ml Hypnorm (Janssen) and i.m. 0.8 ml Dormicum (Roche). time points were studied. Three days after the denudation EC-SOD or LacZ adenovirus gene transfer (3×10⁹ pfu/kg) was performed to the abdominal aortic segment using a local drug delivery catheter (Dispatch catheter, Boston Scientific). Serum samples were collected before the gene transfer, three and seven days after the gene transfer, and at the end of study. Two weeks (EC-SOD n=10 and LacZ n=10) or four weeks (EC-SOD n=4 and LacZ n=4) after the gene transfer the animals were sacrificed.

Multiple tissue samples were collected to determine the biodistribution of adenovirus, as described above. Gene transfer site and adjacent segments of abdominal aorta from renal arteries to bifurcation point were analyzed histologically to determine the effect of adenoviral EC-SOD gene transfer on neointima formation. Aortic sections were obtained at 3 sites: the segment for gene transfer, a segment 2 cm proximal to, and a segment 2 cm distal from the gene transfer site. After removal of the vessel segments, the specimen were flushed gently with saline and divided into three equal parts. One part was immersion-fixed in 4% paraformaldehyde/15% sucrose (pH 7.4) for 4 h, rinsed overnight in 15% sucrose (pH 7.4) and embedded in paraffin. Another part was fixed in 4% paraformaldehyde/15% sucrose (pH 7.4) for 10 min, rinsed in PBS, embedded in OCT compound (Miles Scientific, Elkhart) and stored at −70° C. until subsequent analysis of gene transfer efficiency by X-gal staining for 6 h at +37° C. (LacZ transfected animals). The third part was snap-frozen and further stored at −70° C. until RT-PCR analysis (EC-SOD transfected animals). Neointima formation was quantified after hematoxylinleosin staining using Image-pro plus software with Olympus AX70 microscope (Olympus Optical) as previously described (Hiltunen M O, Laitinen M, Turunen M P, Jeltsch M, Hartikainen J, Rissanen T T, Laukkanen J, Niemi M, Kossila M, Hakkinen T P, Kivela A, Enholm B, Mansukoski H, Turunen A M, Alitalo K, Yla-Herttuala S: Intravascular adenovirus-mediated VEGF-C gene transfer reduces neointima formation in balloon-denuded rabbit aorta. Circulation 2000; 102:2262-2268).

Following antibodies were used to study the aortic segments: CD31 (endothelium, dilution 1:50, DAKO) verified with vWF, RAM 11 (macrophages, dilution 1:200, DAKO), HHF35 (SMC, dilution 1:50, DAKO), p67phox (NADPH oxidase, dilution 1:100, Transduction Laboratories) eNOS (dilution 1:25, Transduction Laboratories), iNOS (dilution 1:50, Transduction Laboratories), VEGF-A (dilution 1:100, Santa Cruz), VEGF-C (dilution 1:100, Santa Cruz), NF-kappaB (dilution 1:50, Transduction Laboratories). Avidin-biotin-horseradish peroxidase system was used for signal detection (Vector Elite Kit). Apoptosis was detected using ApopTag kit (Intergen) according to the manufacturer's instructions.

One of the landmarks of atherosclerosis is SMC accumulation to the lesion, which is thought to be due to lipid accumulation, oxidized LDL and expression of growth factors. Also, SMC accumulation is a common consequence also after balloon angioplasty causing neointima formation within six months after the procedure (Bittl J A: Advances in coronary angioplasty N. Engl. J. Med. 1996; 335: 1290-1302). Neointima formation in EC-SOD animals (intima-media ratio of 0.09±0.05) was significantly (p<0.001) reduced as compared to LacZ controls (0.32±0.14). Interestingly, inhibition of neointima formation was observed not only in the gene transfer site but also in the flanking segments of the abdominal aorta suggesting more widespread effect on prevention of restenosis. The four weeks time point showed 0.13±0.02 intima-media ratio for EC-SOD group and 0.54±020 for LacZ control group (p<0.001) indicating prolonged protection against intimal hyperplasia The inhibition of neointima formation was similarly extended outside of the gene transfer site as at two weeks time point

Since the magnitude of neointima formation is dependent on the degree of balloon injury to the vessel wall we determined the integrity of internal elastic lamina (IEL) in aortic samples from each experimental group. The measurement showed 4±6% damage to IEL in both EC-SOD and in LacZ control groups at the two weeks time point and 5±6% and 2±2% (P=N.S.) damage at the four weeks time point, respectively, indicating no differences in vessel wall injury between the groups.

Previous reports have shown that restenosis is effectively inhibited by inducing endothelial cell growth with growth factors like vascular endothelial growth factor (VEGF) or with genes related to nitric oxide (NO) production (Yla-Herttuala S, Martin J F: Cardiovascular gene therapy. Lancet; 2000; 355: 213-222). Aortic sections stained with CD31 for endothelial cells showed 86±13% recovery of vessel endothelium after denudation in EC-SOD group at two weeks time point whereas in LacZ control group the endothelial recovery was only 21±13% showing a significant difference (p<0.001). Immunohistological analysis of factors which could be involved in this effect (eNOS, iNOS, VEGF-A, VEGF-C, and NF-kappaB) showed no difference between EC-SOD and LacZ control groups. The endothelial recovery of the control samples reached EC-SOD group at four weeks time point being 88±13% for EC-SOD and 81±19% for LacZ control group.

Activated macrophages are known to secrete many cytoeines and growth factors, such as interleukin-1, platelet derived growth factor, and insulin-like growth factor 1, which induce SMC proliferation and participate in neointima formation (Ross R: The pathogenesis of atherosclerosis: a perspective for the 1990s. Nature 1993;362:801-809, Libby P, Schwartz 15 D, Brogi E, Tanaka H, Clinton S K: A cascade model for restenosis. A special case of atherosclerosis progression. Circulation 1992;86: III47-III52). RAM-11 immunostaining for macrophages showed significantly (p<0.001) decreased macrophage infiltration in neointima in EC-SOD group at both time points. The EC-SOD group showed 10-fold decreased accumulation of macrophages as compared to LacZ controls two weeks after the gene transfer and 20-fold decreased accumulation four weeks after the gene transfer. NADPH oxidase, which is reported to be up-regulated in balloon-injured rabbit aortas (West N, Guzik T, Black E, Channon K: Enhanced superoxide production in experimental venous bypass graft intimal hyperplasia: role of NAD(P)H oxidase. Arterioscler. Thromb. Vasc. Biol 2001;21:189-194), was detected in the same areas as macrophages (FIGS. 2i,j and 3i,j). SMC α-actin staining indicated that at the two weeks time point intimal thickening was mostly caused accumulation of SMCs. At the four weeks time point the accumulated macrophages in the neointima had already formed foam cells.

Apoptosis occurs frequently in vascular remodeling both in normal vessel wall development and in atherosclerotic lesions. Apoptosis was remarkably higher in LacZ control vessels than in EC-SOD group at 2 weeks time point but the difference was not present at 4 weeks time point. Autopsy and clinical chemistry analysis showed no toxicity in neither of the animal groups.

In the present study we have shown that adenovirus mediated EC-SOD gene transfer attenuated denudation derived total SOD loss in plasma and resulted in a significant (p<0.001) inhibition of neointima formation in ballooned rabbit aortas for prolonged period. The anti-restenotic effect of EC-SOD may be based on the antioxidative nature of the enzyme. Recently it was shown that lucigenin reductase activity, which reflects the amount of O₂⁻, is increased immediately after injury to rabbit artrial rings. The oxidative stress was highest within a few minutes after injury and was gradually decreased showing only minor lucigenin reductase activity at 14 day time point suggesting that oxidative stress is an early event after endothelial denudation (Azevedo L C, Pedro M A, Souza L C, de Souza H P, Janiszewski M, da Luz P L, Laurindo F R: Oxidative stress as a signaling mechanism of the vascular response to injury: the redox hypothesis of restenosis. Cardiovasc. Res. 2000;47: 436-445). The therapeutical effect was extended affecting both the gene transfer site and adjacent abdominal aorta segments from renal arteries to bifurcation point. This may be due to the ability of plasma EC-SOD to bind to heparan sulfate proteoglycans on the glycogalyx of the cell membranes (Karlsson K, Marklund S L: Heparin-induced release of extracellular superoxide dismutase to human blood plasma. Biochem J. 1987; 242:55-59). Previous studies with ischemic rabbit models are in agreement with current finding showing that systemic adenovirus mediated EC-SOD gene transfer targeted to liver prevents ischemia-reperfusion injury in coronary vessels (Li Q, Bolli R, Qiu Y, Tang X L, Murphree S S, French B A: Gene therapy with extracellular superoxide dismutase attenuates myocardial stunning in conscious rabbits. Circulation 1998;98: 1438-1448, and Li Q, Bolli R, Qiu Y, Tang X L, Guo Y, French B A: Gene therapy with extracellular superoxide dismutase protects conscious rabbits against myocardial infarction. Circulation. 2001;103: 1893-1898), suggesting that EC-SOD synthesized even in different organs can have a protective systemic effect.

EC-SOD transduced aortas had significantly increased endothelium recovery and reduced macrophage accumulation into vessel wall. Both of these alone have been reported to reduce neointima growth in animal models (Asahara T, Chen D, Tsurumi Y, Kearney M, Rossow S, Passeri J, Symes J F, Isner J M: Accelerated restitution of endothelial integrity and endothelium-dependent function after phVEGF165 gene transfer, Circulation 1996; 94: 3291-3302, and Ross R: Atherosclerosis-an inflammatory disease, N. Engl. J. Med. 1999;340: 115-126). The reduced infiltration of macrophages suggests anti-inflammatory role for EC-SOD in addition to antioxidative and anti-apoptototic roles which we have shown in previous studies (Laukkanen M O, Lehtolainen P, Turunen P, Aittomaki S, Oikari P, Marklund S L, Yla-Herttuala S: Rabbit extracellular superoxide dismutase: expression and effect on LDL oxidation. Gene 2000; 254: 173-179, and Laukkanen, M. O., Leppanen, P., Turunen, P, Tuomisto, T, Naarala, J, and Yla-Herttuala, S. EC-SOD gene therapy reduces paracetamol-induced liver damage in mice. J. Gene Med 2001; 3: 321-325).

EC-SOD gene transfer reduced apoptosis at two weeks time point. The exact role for apoptosis in intimal hyperplasia is unknown but early stage apoptosis has been suggested to stimulate restenosis after ballooning by provoking wound healing process whereas late stage apoptosis may inhibit neointima formation by balancing the amount of proliferating cells and rate of neointimal SMC death (Miwa K, Asano M, Horai R, Iwakura Y, Nagata S, Suda T: Caspase 1-independent IL-1beta release and inflammation induced by the apoptosis inducer Fas ligand. Nat. Med 1998;4: 1287-1292, and Walsh K, Smith R C, Kim H S: Vascular cell apoptosis in remodeling, restenosis, and plaque rupture. Circ. Res. 2000;87:184-188).

In conclusion, our results indicates that local catheter-mediated delivery of EC-SOD adenoviruses can reduce restenosis in rabbits and is a useful tool for the prevention of this condition in humans.

In FIG. 1 panels A, C, B, G, I, K, and M represent EC-SOD group and panels B, D, F, H, J, L, and N represents the Lac Z group. Arrows indicate the location of internal lamina A and B. C and D internal elastic lamina determination; E and F endothelium staining (CD-31); G and H BrdU staining; I and J macrophage staining (Ram 11); K and L NADPH-oxidase staining (p67phox); M and N SMC staining (EF-35). Intima/media area ratio, cell proliferation, and accumulation of macrophages were significantly decreased in EC-SOD groups as compared to LacZ controls. Measurements of internal elastic lamina showed similar damage in both groups.

1-49. (Canceled)

50. A method for treating and/or preventing restenosis in a mammal, comprising administering to the mammal a composition comprising a nucleic acid encoding extracellular superoxide dismutase in an amount sufficient to reduce and/or prevent restenosis.

51. A method according to claim 50, wherein the composition is administered by local or systemic delivery.

52. A method according to claim 50, wherein the nucleic acid is present in a biologically compatible medium in naked form.

53. A method according to claim 50, wherein the nucleic acid is in a viral vector selected from the group consisting of retrovirus, Sendai virus, adeno-associated virus and adenovirus.

54. A method according claim 50, wherein the nucleic acid is present in a liposome.

55. A method according to claims 52, wherein the biologically compatible medium is a biostable polymer, a bioabsorbable polymer, a biomolecule, a hydrogel polymer or fibrin.

56. A method according to claim 50, wherein the step of administering the composition is repeated at least once.

57. A method according to claim 50, wherein the mammal is a human.

58. A method for treating and/or preventing blood vessel thickening in a mammal, comprising administering to the mammal a composition comprising a nucleic acid encoding extracellular superoxide dismutase in an amount sufficient to reduce and/or prevent blood vessel thickening.

59. A method according to claim 58, wherein the composition is administered by local or systemic delivery.

60. A method according to claim 58, wherein the nucleic acid is present in a biologically compatible medium in naked form.

61. A method according to claim 58, wherein the nucleic acid is in a viral vector selected from the group consisting of retrovirus, Sendai virus, adeno-associated virus and adenovirus.

62. A method according claim 58, wherein the nucleic acid is present in a liposome.

63. A method according to claims 60, wherein the biologically compatible medium is a biostable polymer, a bioabsorbable polymer, a biomolecule, a hydrogel polymer or fibrin.

64. A method according to claim 58, wherein the step of administering the composition is repeated at least once.

65. A method according to claim 58, wherein the mammal is a human.

66. A method for treating and/or preventing restenosis in a mammal, comprising administering to the mammal a composition comprising an extracellular superoxide dismutase in an amount sufficient to reduce and/or prevent restenosis.

67. A method according to claim 66, wherein the composition is administered by local or systemic delivery.

68. A method according to claim 66, wherein the nucleic acid is present in a biologically compatible medium in naked form.

69. A method according to claim 66, wherein the nucleic acid is in a viral vector selected from the group consisting of retrovirus, Sendai virus, adeno-associated virus and adenovirus.

70. A method according claim 66, wherein the nucleic acid is present in a liposome.

71. A method according to claims 68, wherein the biologically compatible medium is a biostable polymer, a bioabsorbable polymer, a biomolecule, a hydrogel polymer or fibrin.

72. A method according to claim 66, wherein the step of administering the composition is repeated at least once.

73. A method according to claim 66, wherein the mammal is a human.

74. A method for treating and/or preventing blood vessel thickening in a mammal, comprising administering to the mammal a composition comprising an extracellular superoxide dismutase in an amount sufficient to reduce and/or prevent blood vessel thickening.

75. A method according to claim 74, wherein the composition is administered by local or systemic delivery.

76. A method according to claim 74, wherein the nucleic acid is present in a biologically compatible medium in naked form.

77. A method according to claim 74, wherein the nucleic acid is in a viral vector selected from the group consisting of retrovirus, Sendai virus, adeno-associated virus and adenovirus.

78. A method according claim 74, wherein the nucleic acid is present in a liposome.

79. A method according to claims 76, wherein the biologically compatible medium is a biostable polymer, a bioabsorbable polymer, a biomolecule, a hydrogel polymer or fibrin.

80. A method according to claim 74, wherein the step of administering the composition is repeated at least once.

81. A method according to claim 74, wherein the mammal is a human.

82. A method for treating and/or preventing restenosis in a mammal, comprising administering to the mammal a composition comprising a nucleic acid and a biologically compatible medium in an amount sufficient to reduce and/or prevent restenosis, wherein the nucleic acid encodes a translation or transcription product that leads to the production of extracellular superoxide dismutase protein.

83. A method according to claim 82, wherein the composition is administered by local or systemic delivery.

84. A method according to claim 82, wherein the nucleic acid is present in naked form.

85. A method according to claim 82, wherein the nucleic acid is in a viral vector selected from the group consisting of retrovirus, Sendai virus, adeno-associated virus and adenovirus.

86. A method according claim 82, wherein the nucleic acid is present in a liposome.

87. A method according to claims 82, wherein the biologically compatible medium is a biostable polymer, a bioabsorbable polymer, a biomolecule, a hydrogel polymer or fibrin.

88. A method according to claim 82, wherein the step of administering the composition is repeated at least once.

89. A method according to claim 82, wherein the mammal is a human.

90. A method for treating and/or preventing blood vessel thickening in a mammal, comprising administering to the mammal a composition comprising a nucleic acid and a biologically compatible medium in an amount sufficient to reduce and/or prevent blood vessel thickening, wherein the nucleic acid encodes a translation or transcription product that leads to the production of extracellular superoxide dismutase.

91. A method according to claim 90, wherein the composition is administered by local or systemic delivery.

92. A method according to claim 90, wherein the nucleic acid is in naked form.

93. A method according to claim 90, wherein the nucleic acid is in a viral vector selected from the group consisting of retrovirus, Sendai virus, adeno-associated virus and adenovirus.

94. A method according claim 90, wherein the nucleic acid is present in a liposome.

95. A method according to claims 90, wherein the biologically compatible medium is a biostable polymer, a bioabsorbable polymer, a biomolecule, a hydrogel polymer or fibrin.

96. A method according to claim 90, wherein the step of administering the composition is repeated at least once.

97. A method according to claim 90, wherein the mammal is a human.

98. A method for decreasing macrophage accumulation in a mammal, comprising administering to the mammal a composition in an amount sufficient to decrease macrophage accumulation, wherein the composition comprises a nucleic acid encoding extracellular superoxide dismutase, an extracellular superoxide dismutase protein, or a nucleic acid present in a biologically compatible medium, wherein the nucleic acid encodes a translation or transcription product that leads to the production of extracellular superoxide dismutase protein.

99. A method according to claim 98, wherein the composition is administered by local or systemic delivery.

100. A method according to claim 98, wherein the nucleic acid present in a biologically compatible medium is in naked form.

101. A method according to claim 98, wherein the nucleic acid is in a viral vector selected from the group consisting of retrovirus, Sendai virus, adeno-associated virus and adenovirus.

102. A method according claim 98, wherein the nucleic acid is present in a liposome.

103. A method according to claim 98, wherein the biologically compatible medium is a biostable polymer, a bioabsorbable polymer, a biomolecule, a hydrogel polymer or fibrin.

104. A method according to claim 98, wherein the step of administering the composition is repeated at least once.

105. A method according to claim 98, wherein the mammal is a human.

106. A method for increasing endothelial cell growth in a mammal, comprising administering to the mammal a composition in an amount sufficient to increase endothelial cell growth, wherein the composition comprises a nucleic acid encoding extracellular superoxide dismutase, an extracellular superoxide dismutase protein, or a nucleic acid present in a biologically compatible medium, wherein the nucleic acid encodes a translation or transcription product that leads to the production of extracellular superoxide dismutase protein.

107. A method according to claim 106, wherein the composition is administered by local or systemic delivery.

108. A method according to claim 106, wherein the nucleic acid present in a biologically compatible medium is in naked form.

109. A method according to claim 106, wherein the nucleic acid is in a viral vector selected from the group consisting of retrovirus, Sendai virus, adeno-associated virus and adenovirus.

110. A method according claim 106, wherein the nucleic acid is present in a liposome.

111. A method according to claims 106, wherein the biologically compatible medium is a biostable polymer, a bioabsorbable polymer, a biomolecule, a hydrogel polymer or fibrin.

112. A method according to claim 106, wherein the step of administering the composition is repeated at least once.

113. A method according to claim 106, wherein the mammal is a human.

114. A method for inhibition of hyperplastic connective tissue growth and/or promoting endothelialisation in a mammal, comprising administering to the mammal a composition in an amount sufficient to inhibit hyperplastic connective tissue growth and/or promote endothelialisation, wherein the composition comprises a nucleic acid encoding extracellular superoxide dismutase, an extracellular superoxide dismutase protein, or a nucleic acid present in a biologically compatible medium, wherein the nucleic acid encodes a translation or transcription product that leads to the production of extracellular superoxide dismutase protein.

115. A method according to claim 114, wherein the composition is administered by local or systemic delivery.

116. A method according to claim 114, wherein the nucleic acid present in a biologically compatible medium is in naked form.

117. A method according to claim 114, wherein the nucleic acid is in a viral vector selected from the group consisting of retrovirus, Sendai virus, adeno-associated virus and adenovirus.

118. A method according claim 114 wherein the nucleic acid is present in a liposome.

119. A method according to claims 114, wherein the biologically compatible medium is a biostable polymer, a bioabsorbable polymer, a biomolecule, a hydrogel polymer or fibrin.

120. A method according to claim 114, wherein the step of administering the composition is repeated at least once.

121. A method according to claim 114, wherein the mammal is a human.

122. A method for inhibiting hyperplastic connective tissue growth, or fibromuscular formation and/or promoting endothelialisation in a mammal, comprising administering to the mammal a composition in an amount sufficient to inhibit hyperplastic connective tissue growth, or fibromuscular formation, and/or promote endothelialisation, 7 wherein the composition comprises a nucleic acid encoding extracellular superoxide dismutase, an extracellular superoxide dismutase protein, or a nucleic acid present in a biologically compatible medium, wherein the nucleic acid encodes a translation or transcription product that leads to the production of extracellular superoxide dismutase protein.

123. A method according to claim 122, wherein the composition is administered by local or systemic delivery.

124. A method according to claim 122, wherein the nucleic acid in a biologically compatible medium is present in naked form.

125. A method according to claim 122, wherein the nucleic acid is in a viral vector selected from the group consisting of retrovirus, Sendai virus, adeno-associated virus and adenovirus.

126. A method according claim 122, wherein the nucleic acid is present in a liposome.

127. A method according to claim 122, wherein the biologically compatible medium is a biostable polymer, a bioabsorbable polymer, a biomolecule, a hydrogel polymer or fibrin.

128. A method according to claim 122, wherein the step of administering the composition is repeated at least once.

129. A method according to claim 122, wherein the mammal is a human.


SEQUENCE LISTING (4 sequences)

SEQ ID NO: 1 - DNA, 21 bp, Artificial Sequence
  primer
gatgctggcgttggtgtgctc

SEQ ID NO: 2 - DNA, 21 bp, Artificial Sequence
  primer
gcacggccagcgggttgtagt

SEQ ID NO: 3 - DNA, 21 bp, Artificial Sequence
  primer
ggatgttgcaagtgaccaggc

SEQ ID NO: 4 - DNA, 21 bp, Artificial Sequence
  primer
gcacggccagcgggttgtagt